(19) United States
(12) Patent Application Publication
(10) Pub. No.: US 2011/0091945 A1
(43) Pub. Date: Apr. 21, 2011

(54) METHODS OF INCREASING BIOMASS PRODUCTIVITY, LIPID INDUCTION, AND CONTROLLING METABOLITES IN ALGAE FOR PRODUCTION OF BIOFUELS USING BIOCHEMICAL STIMULANTS

(75) Inventors: Keshav Das, Athens, GA (US); Ryan W. Hunt, Athens, GA (US); Senthil Chinnasamy, Athens, GA (US); Ronald Claxton, Winterville, GA (US); Patrick Raber, Dalton, GA (US)

(73) Assignee: University of Georgia Research Foundation, Inc., Athens, GA (US)

(21) Appl. No.: 12/907,206

(22) Filed: Oct. 19, 2010

Related U.S. Application Data
(60) Provisional application No. 61/253,535, filed on Oct. 21, 2009.
(60) Provisional application No. 61/362,777, filed on Jul. 9, 2010.

(51) Int. Cl. C12P 7/64; C12P 1/00

(52) U.S. Cl. 435/134; 435/41

(57) ABSTRACT

The present disclosure provides methods of enhancing the biofuel potential of an algal culture, the ability of an algal culture to provide a biofuel such as a lipid or to be processed to a biofuel, the method comprising: contacting an algal culture with a composition selected to enhance the biofuel potential of an algal culture; and allowing the algal culture to incubate to the point where the potential of the algal culture to provide a biofuel product or be processed to a biofuel product is enhanced compared to when the algal culture is not in contact with the composition. The selected algal species can be a species of a genus selected from the group consisting of: Gloeocystis, Limnothrix, Scenedesmus, Chlorococcum, Chlorella, Anabaena, Chlamydomonas, Botryococcus, Cricosphaera, Spirulina, Nannochloris, Dunaliella, Phaeodactylum, Pleurochrysis, Tetraselmis, or any combination thereof, one suitable species being Chlorella sorokiniana. In some embodiments, the composition selected to enhance the biofuel potential of an algal culture can be a pesticide such as, but not limited to, malathion (2-(dimethoxyphosphinothioylthio)butanedioic acid diethyl ester).

### CROSS-REFERENCE TO RELATED APPLICATIONS

This application claims priority to U.S. Provisional Patent Application Ser. No. 61/253,535, entitled “BIOCHEMICAL STIMULANTS FOR ALGAE, METHODS OF INCREASING BIOMASS PRODUCTIVITY OF ALGAE, METHODS OF CONTROLLING METABOLITES IN ALGAE” filed on Oct. 21, 2009, and to U.S. Provisional Patent Application Ser. No. 61/362,777, entitled “LIPID INDUCTION IN ALGAE USING STRESS STIMULANTS FOR PRODUCTION OF BIOFUELS” filed on Jul. 9, 2010 the entireties of which are hereby incorporated by reference.

### FEDERAL SPONSORSHIP

This invention was made with Government support under Contract/Grant No. DE-FE36-08GO88144, awarded by the Department of Energy. The Government has certain rights in this invention.

### TECHNICAL FIELD

The present disclosure is generally related to methods of enhancing the lipid content of algae using biochemical stimulants.

### BACKGROUND

Algae are considered to be potential candidates for production of advanced biofuels in view of growing global energy concerns. They are an attractive option over terrestrial crops due to their ability to grow fast, produce large quantities of lipids, carbohydrates and proteins, thrive in poor quality waters, sequester and recycle carbon dioxide from industrial flue gases and remove pollutants from industrial, agricultural and municipal wastewaters (Hu et al., (2008) Plant J. 54: 621-639). Microalgae offer great promise to contribute a significant portion of the renewable fuels that will be required to meet the U.S. biofuel production target of 36 billion gallons by 2022, out of which 21 billion gallons should be from advanced biofuels as mandated in the Energy Independence and Security Act of 2007 under the Renewable Fuels Standard. To meet 100% of the mandated requirement of advanced biofuels, about 4.3 million ha of algal ponds are needed.

Though large-scale cultivation of algae for food, feed and nutraceutical is a proven technology, there are significant challenges in producing algal biomass specifically for production of biofuels. Studies conducted in the past suggested that growth and lipid production are mutually exclusive. If the growth rate is high, lipid content of algae is found to be low. Nitrogen starvation and genetic engineering are the two areas currently being considered to improve lipid production in algae.

The world-wide desire to progress past fossil fuels into carbon neutral and carbon negative fuels has led many research teams to explore the potential of microalgae for biofuel and bioenergy applications. Most traditional studies to increase biomass productivities have focused on strain selection, nitrogen and phosphorus nutrient uptake and CO₂ supplementation. Traditional cultivation and manipulation of biological systems have consisted of natural selection and genetic engineering modalities. Recently, metabolic engineering and synthetic biology are gaining wide attention from the scientific community due to the immense potential in living systems especially microbes for medical, agricultural, industrial and environmental applications. However, genetic manipulation leads to inheritable changes in a species that might affect the ecosystem adversely when used for environmental and agricultural applications. The enhancement of microalgae cultivation using various biostimulants such as growth promoters, phytohormones, and micronutrients was reported as early as the 1930′s (Brannon & Bartsch (1939). J. Bot. 26: 179-269; Brian et al., (1954) J. Sci Food Ag. 5: 602-612; Liu et al., (2008) Bioresource Technol. 99:4717-4722; Lee & Bartlett (1976) Vermont Agri. Exp. Stn. J. 353: 876-879.). However, as of recently, there has been little work in this field with respect to biofuels and other value-added products from microalgae.

Biochemical stimulants offer potential to enhance the yields and productivities in microalgae cultivation. The average biomass productivity reported in the literature for the conventional commercial scale open pond system is 20 g/m²/d with biomass concentrations of 0.1 g/L/d. This translates into approximately 30 t/acre/year. Enhancing the biomass productivity per acre per year from 30 t/acre/year to 60 t/acre/year will greatly reduce the cost of production of biomass and increase the economic viability of biofuels production from algae. In addition, the use of biostimulants in commercial cultivation of algae can significantly increase the profitability of industries producing algal biomass for production of food, feed, nutraceutical and pharmaceutical products.

### SUMMARY

The present disclosure, therefore, encompasses embodiments of methods of enhancing the biofuel potential of an algal culture, the method comprising: providing a culture of at least one algal species; contacting the algal culture with a composition selected to enhance the biofuel potential of an algal culture; and allowing the algal culture to incubate for, whereby the potential of the algal culture to provide a biofuel product or be processed to a biofuel product is enhanced compared to when the algal culture is not in contact with the composition.

In embodiments of the methods of the disclosure, the alga species can be a species of a genus selected from the group consisting of: Gloeocystis, Limnothrix, Scenedesmus, Chlorococcum, Chlorella, Anabaena, Chlamydomonas, Botryococcus, Cricosphaera, Spirulina, Nannochloris, Dunaliella, Phaeodactylum, Pleurochrysis, Tetraselmis, and any combination thereof.

In certain embodiments of the methods of the disclosure, the alga species can be Chlorella sorokiniana.

In embodiments of the methods of the disclosure, the composition selected to enhance the biofuel potential of an algal culture can be selected from the group consisting of: an auxin, a phytohormone, a cytokinin, a cytokinin like compound, a growth promoter, a micronutrient, and any combination thereof.

In some embodiments of the methods of the disclosure, the composition selected to enhance the biofuel potential of an algal culture can be selected from the group consisting of: phenyl acetic acid indole-butryic acid, naphthalene acetic acid, gibberellic acid, zeatin, thidiazuron, humic acid, kelp extract, methanol, iron chloride, and any combination thereof.

In embodiments of the methods of the disclosure, the enhanced biofuel potential can be the lipid content of the algal culture.

In some embodiments of the methods of the disclosure, the composition selected to enhance the biofuel potential of an algal culture can be a pesticide.

In one embodiment of the methods of the disclosure, the pesticide can be malathion (2-(dimethoxyphosphinothioylthio)butanedioic acid diethyl ester).

### BRIEF DESCRIPTION OF THE DRAWINGS

Further aspects of the present disclosure will be more readily appreciated upon review of the detailed description of its various embodiments, described below, when taken in conjunction with the accompanying drawings.

The drawings are described in detail in the description and examples below.

The details of some exemplary embodiments of the methods and systems of the present disclosure are set forth in the description below. Other features, objects, and advantages of the disclosure will be apparent to one of skill in the art upon examination of the following description, drawings, examples and claims. It is intended that all such additional systems, methods, features, and advantages be included within this description, be within the scope of the present disclosure, and be protected by the accompanying claims.

FIG. 1 illustrates the chemical structures of auxins.

FIG. 2 illustrates the chemical structure of gibberellic acid, Zeatin, Thidiazuron, and humic acid.

FIG. 3 is a graph illustrating the Effect of various biostimulants on the biomass productivity of C. sorokiniana.

FIG. 4 is a graph illustrating changes in biomass productivity (%) in various treatments containing biostimulants over control.

FIG. 5 is a graph illustrating the effect of various biostimulants on chl a content of C. sorokiniana.

FIG. 6 is a graph illustrating the effect of various biostimulants on chl b content of C.sorokiniana.

FIG. 7 is a graph illustrating the effect of various biostimulants on total chlorophyll content of C.sorokiniana.

FIG. 8 is a graph illustrating changes in total chlorophyll in various treatments containing biostimulants over control in C. sorokiniana.

FIG. 9 is a graph illustrating changes in total chlorophyll and biomass in various treatments containing biostimulants over control on day 5 in C. sorokiniana.

FIG. 10 is a graph illustrating is a graph illustrating changes in total chlorophyll and biomass in various treatments containing biostimulants over control on day 10 in C.sorokiniana.

FIG. 11 is a graph illustrating the effect of various biostimulants on total carbohydrates content in C.sorokiniana.

FIG. 12 is a graph illustrating changes in total carbohydrates in various treatments containing biostimulants over control on day 5 and 10 in C. sorokiniana.

FIG. 13 is a graph illustrating changes in total carbohydrates and biomass in various treatments containing biostimulants over control on day 5 in C. sorokiniana.

FIG. 14 is a graph illustrating changes in total carbohydrates and biomass in various treatments containing biostimulants over control on day 10 in C. sorokiniana.

FIG. 15 is a graph illustrating changes in total carbohydrates and chlorophyll in various treatments containing biostimulants over control on day 10 in C. sorokiniana.

FIG. 16 is a graph illustrating time dependent changes in biomass production in Chlorella sorokiniana under different concentrations of malathion. C-Control (BG11 only); T1: 70 mg of malathion L⁻¹; T2: 140 mg of malathion L⁻¹; T3: 280 mg of malathion L⁻¹. Results indicated that there was no growth observed in the treatments T3 and T4 (560 mg of malathion L⁻¹). Both the treatments proved lethal. Values are means±S.D with n=3.

FIG. 17 is a graph illustrating time dependent changes in chlorophyll a production in Chlorella sorokiniana under different concentrations of malathion. C-Control (BG11 only); T1: 70 mg of malathion L⁻¹; T2: 140 mg of malathion L⁻¹; T3: 280 mg of malathion L⁻¹. Results indicated that there was no growth observed in the treatments T3 and T4 (560 mg of malathion L⁻¹). Both the treatments proved lethal. Values are means±S.D with n=3.

FIG. 18 is a graph illustrating pH changes in the growth medium for Chlorella sorokiniana with different concentrations of malathion. C-Control (BG11 only); T1: 70 mg of malathion L⁻¹; T2: 140 mg of malathion L⁻¹; T3: 280 mg of malathion L⁻¹. Results indicated that there was no growth observed in the treatments T3 and T4 (560 mg of malathion L⁻¹). Both the treatments proved lethal. Values are means±S.D with n=3.

FIG. 19 is a graph illustrating time dependent changes in lipid content in Chlorella sorokiniana under different concentrations of malathion. C-Control (BG11 only); T1: 70 mg of malathion L⁻¹; T2: 140 mg of malathion L⁻¹; T3: 280 mg of malathion L⁻¹. Results indicated that there was no growth observed in the treatments T3 and T4 (560 mg of malathion L⁻¹) on day 12. Both the treatments proved lethal. Values are means±S.D with n=3.

FIG. 20 is a graph illustrating changes in the lipid growth medium for Chlorella sorokiniana with different concentrations of malathion. C-Control (BG11 only); T1: 70 mg of malathion L⁻¹; T2: 140 mg of malathion L⁻¹; T3: 280 mg of malathion L⁻¹. Results indicated that there was no growth observed in the treatments T3 and T4 (560 mg of malathion L⁻¹). Both the treatments proved lethal. Values are means±S.D with n=3.

FIG. 21 is a graph illustrating changes in the potential maximum quantum yield (Fv/Fm) of photosystem II observed in Chlorella sorokiniana under different concentrations of malathion. C-Control (BG11 only); T1: 70 mg of malathion L⁻¹; T2: 140 mg of malathion L⁻¹; T3: 280 mg of malathion L⁻¹. Results indicated that there was no growth observed in the treatments T3 and T4 (560 mg of malathion L⁻¹). Both the treatments proved lethal. Values are means±S.D with n=3.

FIGS. 22A-22D is a series of graphs illustrating changes in (FIG. 22A) effective photosynthetic yield, (FIG. 22B) electron transfer rate, (FIG. 22C) photochemical quenching and (FIG. 22D) non photochemical quenching observed in Chlorella sorokiniana under different concentrations of malathion. C-Control (BG11 only); T1: 70 mg of malathion L⁻¹; T2: 140 mg of malathion L⁻¹; T3: 280 mg of malathion L⁻¹. Results indicated that there was no growth observed in the treatments T3 and T4 (560 mg of malathion L⁻¹). Both the treatments proved lethal. Values are means±S.D with n=3.

FIG. 23 is a schema showing possible cell responses and mechanisms involved in the pesticide stress caused by malathion in Chlorella sorokiniana

FIG. 24 is a graph illustrating changes in the lipid content of the cells grown in C (control with no malathion), T1 (70 mg malathion L⁻¹) and T2 (140 mg malathion L⁻¹) with respect to day 3. Compared to 97% increase in lipid content observed in the control on day 9, the treatments T1 and T2 recorded 193% and 150% increase, respectively. On day 12 control recorded 287% increase whereas T1 and T2 recorded an increase of 406% and 574%, respectively.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

As used herein, the following terms have the meanings ascribed to them unless specified otherwise. In this disclosure, “comprises,” “comprising,” “containing” and “having” and the like can have the meaning ascribed to them in U.S. Patent law and can mean “ includes,” “including,” and the like; “consisting essentially of or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of or “consists essentially” or the like, when applied to methods and compositions encompassed by the present disclosure have the meaning ascribed in U.S. Patent law and the term is open-ended, allowing for the presence of more than that which is recited so long as basic or novel characteristics of that which is recited is not changed by the presence of more than that which is recited, but excludes prior art embodiments.

Prior to describing the various embodiments, the following definitions are provided and should be used unless otherwise indicated.

### Definitions

In describing and claiming the disclosed subject matter, the following terminology will be used in accordance with the definitions set forth below.

The terms “algae” and “algal cells” as used herein refer to a large and diverse group of simple, typically autotrophic organisms, ranging from unicellular to multicellular forms. They are photosynthetic, like plants, and “simple” because they lack the many distinct organs found in land plants. All true algae have a nucleus enclosed within a membrane and chloroplasts bound in one or more membranes. “Microalgae” or “microphytes” (also referred to as phytoplankton, or planktonic algae) are microscopic algae, typically found in freshwater and marine systems. There are 200,000-800,000 species exist of which about 35,000 species are described.

They are unicellular species which exist individually, or in chains or groups. Depending on the species, their sizes can range from a few micrometers (μm) to a few hundreds of micrometers. Microalgae produce approximately half of the atmospheric oxygen and use simultaneously the greenhouse gas carbon dioxide to grow photoautotrophically. The biodiversity of microalgae is enormous and they represent an almost untapped resource. The chemical composition of microalgae is not an intrinsic constant factor but varies over a wide range, both depending on species and on cultivation conditions. Microalgae such as microphytes constitute the basic foodstuff for numerous aquaculture species, especially filtering bivalves.

The terms “aqueous medium,” “culture medium” and “cultural medium” as used herein refers to an aqueous medium designed to support the growth of algal cells. For example, and by no means intended to be limiting, an aqueous medium includes a natural water source such as a river, stream, lake, brackish water at the boundary between marine water and freshwater environment, or a marine water source. “Culture media” can include, but are not limited to, artificial aqueous media providing nutrients required by the algae, nutrient-rich effluent from agricultural or industrial facilities, land-fill run-off, and the like.

The term “culture system” as used herein refers to a system of water retaining, filtering, heating/cooling, and circulating systems, and structures that are typically employed in the maintenance of a culture medium under conditions suitable for supporting the viability and reproduction of a desired organism(s).

The term “algal culture” as used herein refers to any culture of an algal species or plurality of species.

The term “composition selected to enhance the biofuel potential of an algal culture” as used herein refers to any compound or combination of compounds that when introduced to an algal culture will result in an increase in the biomass product of the culture, or in the amount of at least one constituent component of the algal cells of the culture, or a product released or releasable into the medium of the culture, and which may be used as a source of a biofuel. Such constituent compounds include, but are not limited to, such as a carbohydrate (a sugar, a starch, or the like), a lipid (oil, fat, and the like) or a complex combining one or more such components.

The term “enhanced” as used herein refers to an increase in a parameter or the amount of a compound, plurality of compounds, a polymeric material, and the like produced by an algal cell or algal culture when in the presence of an effective amount of a composition compared to when the algal cell or algal culture is not in the presence of an effective amount of a composition.

The term “proliferation” as used herein refers to algal reproduction and is used in the contexts of cell development and cell division (reproduction). When used in the context of cell division, it refers to growth of cell populations.

The term “viability” as used herein refers to “capacity for survival” and is more specifically used to mean a capacity for living, developing, or germinating under favorable conditions.

The term “biofuel potential” as used herein refers to the capacity of an algal culture to provide a compound or plurality of compounds that may be used as a biofuel such as a biodiesel. In the alternative “biofuel potential” may refer to the capacity of an algal culture to be used as a raw material for conversion to a biofuel by such processes as, but not limited to, pyrolysis, chemical conversion, mechanical extraction, or any combination thereof. it is further contemplated that the “biofuel potential” of an algal culture may refer to such as the biomass in units of weight, lipid content, carbon content, carbohydrate content, and the like and which may be an indirect measurement of the potential amount of algal-sourced raw material for use in the production of a biofuel.

Unless otherwise defined, all other technical and scientific terms used herein have the same meaning as commonly understood by one of ordinary skill in the art of biology. Although methods and materials similar or equivalent to those described herein can be used in the practice or testing of the present invention, suitable methods and materials are described herein.

### Discussion

The present disclosure encompasses methods of increasing biomass production in algae, methods of controlling metabolites in algae, methods of inhibiting chlorophyll synthesis in algae, methods of stimulating growth in algae, and the like. Embodiments of the present disclosure aim at developing biochemical stimulants and combinations thereof capable of enhancing the growth and metabolism of algae. Embodiments of the present disclosure provide a biochemical stimulation that is simple, safe, and eco-friendly technique as compared to conventional genetic and metabolic engineering and is suitable for use in both open ponds and closed photobioreactors.

Embodiments of the present disclosure can be useful in one or more of the following: developing universal mixtures for various species of algae to deliver an optimal dose for maximum biostimulatory effect; developing different blends of biostimulants that promote different metabolite productivities and yields, i.e. predominately stimulating protein, carbohydrate, or lipid synthesis; preventing bacterial and fungal contamination due to the additional biostimulants in the growth medium; and reducing the cost of biostimulants for large scale production by investigating lower dosages, synergistic effects between related and unrelated growth promoters, and adjusting the dosage rate such that the biostimulants are used more efficiently by algae.

Embodiments of the present disclosure provide for methods of increasing biomass production in algae. The methods include introducing one or more biochemical stimulants to a system including algae, and allowing the algae to proliferate for a suitable time period (e.g., 5, 10, or more days). The algae can be selected from, but are not limited to, the genera of Gloeocystis, Limnothrix, Scenedesmus, Chlorococcum, Chlorella, Anabaena, Chlamydomonas, Botryococcus, Cricosphaera, Spirulina, Nannochloris, Dunaliella, Phaeodactylum, Pleurochrysis, Tetraselmis, or any combination thereof. In an embodiment, the alga is Chlorella sorokiniana.

The biochemical stimulants useful in the methods of the disclosure can be, but are not limited to, an auxin, a phytohormone, a cytokinin, a cytokinin-like compound, a growth promoter, a micronutrient, or any combination thereof. In particular, the biochemical stimulant can be, but is not limited to, the group consisting of: phenyl acetic acid indole-butryic acid, naphthalene acetic acid, gibberellic acid, zeatin, thidiazuron, humic acid, kelp extract, methanol, iron chloride, or any combination thereof. The amount of biostimulant can be varied depending on the purpose. Some exemplary concentrations that could be used are described in Example 1.

Although gibberellic acid has been known to have strong species-specific effects, no information could be traced where gibberellic acid has been applied to microalgae for enhancing growth. Despite a wide range of applied dosages, for the screening method, the smallest optimal dosage determined was 10 ppm (Brian et al., (1954) J. Sci. Food Agriculture 5: 602-612).

Another class of phytohormones is cytokinin. Several types of cytokinins and cytokinin-like compounds are known. For the purpose of this experiment the most fundamental form, Zeatin, was used as it was found to be at least 50 times more active than its cis isomer (the structure is shown in FIG. 2) (Schmitz & Skoog (1972) Plant Physiol. 50: 702-705). Its optimal dosage was found to be 10⁻² μM for maximum growth of callus tissue culture from tobacco (Schmitz & Skoog (1972) Plant Physiol. 50:702-705). Its diphenylurea derivative, thidiazuron, though technically not considered as a cytokinin, demonstrates cytokinin-like behavior eliciting a wide array of responses from different species of plants (Murthy et al., (1998) In Vitro Cell. Dev. Biol.-Plant 34: 267-275). Optimal dose chosen for thidiazuron was 1 μM that generated maximum growth in soy callus and expansion of radish cotyledon (Thomas & Katterman (1986) Plant Physiol., 81: 681-683).

Commercial plant growth promoters have often focused on humic acid application to soil to promote soil health and plant growth. It is a heterogeneous mix consisting of a variety of compounds that may be extracted by dilute alkali or acid from organic matter in soil or prepared synthetically from sugars or other similar organic material (Burk et al., (1931) Science 74: 522-524). There exists a range of optimal dosages for microalgae and enormous increases in growth of Chlorella were found at a low concentration of 4 ppm (Toldeo et al., (1979) Hydrobiologia 71: 261-263), whereas 60 ppm was optimal for the growth of the microalga Botrydium (Lee & Bartlett (1976) Vermont Agri. Exp. Stn. J. 353: 876-879). The dosage used in the following experiments was 20 ppm, which was based on the positive effects on tomato plants (Adani et al., (1998) J. Plant Nutrition 21: 561-575) and it falls in between the two optimal dosages found for microalgae.

An underutilized biostimulant that demonstrates a wide range of growth promoting effects are seaweed extracts (Crouch & van Staden (1994) J. Home Consumer Horticulture 1: 21-29). These extracts, like humic acid, are not single compounds, but a heterogeneous mixture of biochemical constituents produced by a variety of methods that may contain the entire gamut of plant growth regulators. The optimal dosage was determined by looking for the minimum dose that offered a substantial enhancement of growth or metabolism, which was 0.2% by weight (Crouch & van Staden (1994) J. Home Consumer Horticulture 1: 21-29). Along with phytohormones, micronutrients such as iron have been found to not only act as a fertilizer to phytoplankton, but also increase the amount of lipids accumulated in microalgal cells. The optimal dosage of FeCl₃ was determined to be 1.2×10⁻⁵ mol Li (Liu et al., (2008) Bioresource Technol. 99: 4717-4722).

The other biochemical stimulant used in the screening study was methanol, which has been found to be highly effective at increasing growth rates similar to an enhancement of high CO₂ concentrations (Kotzabasis et al., (1999) J. Biotechnol. 70: 357-362). The optimal concentration used for this study was 0.5% (v/v) which demonstrated over a 300% increase in the growth rate of the microalgae Scenedesmus obliquus (Theodoridou et al., (2002) Biochim. Biophys. Acta 1573: 189-198).

Naphthalene acetic acid (NAA) at 5 ppm concentration can significantly increase the biomass productivities over two-fold during 5-10 days growth, while inhibiting excessive chlorophyll synthesis per unit biomass. Phenyl acetic acid (PAA) was very effective at stimulating growth by 95% during the first 5 days of growth period, but was less effective during the 5-10 day growth period. The stimulatory response to GA₃ ranked second with respect to overall biomass and an 81% increase on day 10, while inhibiting chlorophyll synthesis throughout the growth period. The application of IBA stimulated biomass growth during the entire 0-10 day growth period, while simultaneously promoting chlorophyll synthesis by 93% v. control. The remaining growth promoting compounds did not achieve a stimulatory effect over 50% at the concentration used in this study in the species Chlorella sorokiniana and thus have not been shortlisted for further growth studies.

Studies in this disclosure lead to developing a range of mixtures of various biostimulants for enhancing biomass productivity and various high value products such as lipids, proteins, carbohydrates and nutraceutical compounds such as beta-carotene and astaxanthin. In addition, the biostimulant mixtures of the present disclosure may play an important role to significantly reduce the cost of production of algal biomass for algal biofuel production in future.

Embodiments of the present disclosure provide for methods of controlling metabolites in algae. The methods can include introducing one or more biochemical stimulants to a system for culturing algae, and allowing the algae to proliferate for a time frame (e.g., 5, 10, or more days). The algae can include the genera described above. In one preferred embodiment, the alga is Chlorella sorokiniana. The biochemical stimulant can include the biochemical stimulates noted above. Additional details regarding the present disclosure are described below.

For example, but not intended to be limiting, embodiments of the present disclosure provide for methods of inhibiting chlorophyll synthesis in algae. The method can include introducing one or more biochemical stimulants to a system including algae and allowing the algae to proliferate for a time frame (e.g., 5, 10, or more days). The algae can include the genera described above. In one preferred embodiment, the alga is Chlorella sorokiniana. The biochemical stimulant can include the biochemical stimulates noted above. Additional details regarding the present disclosure are described below.

Embodiments of the present disclosure provide for methods of stimulating growth in algae. The method can include introducing one or more biochemical stimulants to a system including algae and allowing the algae to proliferate for a time frame (e.g., 5,10, or more days). The algae can include the genera described above. In one preferred embodiment, the alga is Chlorella sorokiniana. The biochemical stimulant can include the biochemical stimulates noted above. Additional details regarding the present disclosure are described below.

Weiner et al., ((2007) Pest Biochem. Physiol. 87: 47-53) reported that the herbicide atrazine at higher concentration (136 mg L⁻¹) significantly increased lipid content in Dunaliella tertiolecta by 101% and reduced protein content by 57% relative to the control. However, there are no reports on pesticide mediated lipid induction in algae. The addition of malathion causes stress to the algal cells and impairs photosynthesis (Lal & Lal (1988) Vol 3. CRC, Boca Raton, Fla., USA; Torres & O'Flaherty (1976) Phycologia 15: 25-36). Excess electrons accumulate in the photosynthetic electron transport chain when the algal cells are subjected to stress. This pesticide induced stress might have induced over-production of reactive oxygen species (superoxide radicals), which may in turn cause inhibition of photosynthesis and damage to membrane lipids, proteins and other macromolecules. This could be the reason why the cells grown under different concentrations of malathion did not show significant growth in the first 6 to 9 days. However, photosynthetic organisms can counteract the toxicity of stress induced free radicals by increasing their antioxidative defense mechanisms that include enzymes such as superoxide dismutase (SOD), catalase (CAT), ascorbate peroxidase (APX) and low molecular weight compounds such as carotenoids, ascorbate, glutathione, flavonoids and tocopherols which act as electron sinks (Kumar et al., (2008) Science of the Total Environment 403: 130-138). The cells respond to the stress by synthesizing C₁₈ fatty acids which are the precursors of neutral lipids (TAG) as it consumes approximately 24 NADPH derived from the electron transport chain. This is twice that required for the synthesis of a carbohydrate or protein molecule of the same mass. C₁₈ fatty acids can act as electron sinks and thus relaxes the over-reduced electron transport chain under stress conditions (Hu et al., (2008) Plant J. 54: 621-639). It has also been reported that the TAG synthesis pathway is usually accompanied with synthesis of carotenoids in algae (Rabbani et al., (1998) Plant Physiol. 116: 1239-1248; Zhekisheva et al., (2002) J. Phycol. 38: 325-331). Carotenoids such as b-carotene, lutein and astaxanthin produced in the carotenoid pathway are esterified with TAG and sequestered into cytosolic lipid bodies during stress (Hu et al., (2008) Plant J. 54: 621-639).

In the current investigation, production of C₁₈ fatty acids and carotenoids in response to pesticide stress could have increased the lipid content in C. sorokiniana. However, the percentage increase in lipid over control was gradually reduced as the cells exhibited reversal of inhibition by day 6 in the treatment with 70 mg malathion L⁻¹ and on day 9 in the treatment with 140 mg malathion L⁻¹. It was evident from the results that the treatments with 70, 140 and 280 mg of malathion L⁻¹ recorded only 42%, 119% and 212% increase on day 9; and on day 12, only the treatment with 140 mg malathion L⁻¹ showed 25% increase in lipid content over control. Though the results indicated significant increase in lipid production in the cells subjected to pesticide stress on day 3, 6 and 9, the lipid productivity was found to be high in the control.

Griffiths & Harrison ((2009) J. Appl. Phycol. 21: 493-507) found that the lipid content does not correlate directly with lipid productivity. They also reported that the species with a high lipid productivity (>60 mg L⁻¹ day⁻¹) can show lipid content as low as 11% dry weight. Similarly they also found that the species with a high lipid content (>40%) vary in lipid productivity as low as 17 mg L⁻¹ day⁻¹. The translation of increased lipid content into an increased lipid productivity is dependent on the degree of growth retardation caused by the stress.

This study also indicates that lipid content has not been a reliable indicator of lipid productivity and the correlation between biomass and lipid productivity was significant. However, this study proved that the pesticide stress-induced lipid production in C. sorokiniana. This can be exploited to improve the lipid productivity of non-oleaginous algal strains with high growth rate to be used as biofuel feedstocks.

It is contemplated, therefore, that pesticides such as malathion can be used as stress stimulants to induce lipids in algae without compromising with biomass productivity. The technology of the present disclosure can be termed as post-harvest lipid induction technology as it advocates growing algae to a higher concentration without any stress and subjecting the harvested cells (in higher concentration) in a reactor with stress stimulants such as malathion at a concentration of 280-540 mg L⁻¹ for a short period to induce lipids. The lipid rich cells after harvesting can be used for further processing. This technology is also suitable for algae cultivated on solid surfaces for treating wastewaters. After developing the biomat/biofilm, the surface of the algal biomat is flooded with stress stimulants for a short period to induce lipids. This technology will be very useful for biofuels production as it can be used for the following: mixed cultures of wild algae growing in ponds and wastewater, filamentous mat of cyanobacterial blooms, microalgal blooms, and non-oleaginous strains. Using this technology lipid can be induced in any weed/wild algae growing in suspended cultures as consortium or solid surfaces as biomat/biofilm.

The present disclosure, therefore, encompasses embodiments of methods of enhancing the biofuel potential of an algal culture, the method comprising: providing a culture of at least one algal species; contacting the algal culture with a composition selected to enhance the biofuel potential of an algal culture; and allowing the algal culture to incubate for, whereby the potential of the algal culture to provide a biofuel product or be processed to a biofuel product is enhanced compared to when the algal culture is not in contact with the biochemical stimulant.

In embodiments of the methods of the disclosure, the alga species can be a species of a genus selected from the group consisting of: Gloeocystis, Limnothrix, Scenedesmus, Chlorococcum, Chlorella, Anabaena, Chlamydomonas, Botryococcus, Cricosphaera, Spirulina, Nannochloris, Dunaliella, Phaeodactylum, Pleurochrysis, Tetraselmis, and any combination thereof.

In certain embodiments of the methods of the disclosure, the alga species can be Chlorella sorokiniana.

In embodiments of the methods of the disclosure, the composition selected to enhance the biofuel potential of an algal culture can be selected from the group consisting of: an auxin, a phytohormone, a cytokinin, a cytokinin like compound, a growth promoter, a micronutrient, and any combination thereof.

In some embodiments of the methods of the disclosure, the composition selected to enhance the biofuel potential of an algal culture can be selected from the group consisting of: phenyl acetic acid indole-butryic acid, naphthalene acetic acid, gibberellic acid, zeatin, thidiazuron, humic acid, kelp extract, methanol, iron chloride, and any combination thereof.

In embodiments of the methods of the disclosure, the enhanced biofuel potential can be selected from the group consisting of: the biomass of the algal culture and the lipid content of the algal culture.

In embodiments of the methods of the disclosure, the composition selected to enhance the biofuel potential of an algal culture can be contacted with the algal culture at a time point within the incubation time period whereby enhancement of the biofuel potential of an algal culture is greater than when the composition is contacted with the algal culture at a different time point within the incubation time period.

In some embodiments of the methods of the disclosure, the composition selected to enhance the biofuel potential of an algal culture can be a pesticide.

In one embodiment of the methods of the disclosure, the pesticide can be malathion (2-(dimethoxyphosphinothioylthio)butanedioic acid diethyl ester).

It should be emphasized that the embodiments of the present disclosure, particularly, any “preferred” embodiments, are merely possible examples of the implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure, and the present disclosure and protected by the following claims.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include ±1%, ±2%, ±3%, ±4%, ±5%, ±6%, ±7%, ±8%, ±9%, or ±10%, or more of the numerical value(s) being modified.

### EXAMPLES

### Example 1

Microalgae Cultivation: Chlorella sorokiniana (UTEX 2805) was obtained from UTEX Culture Collections and maintained in BG11 medium (NaNO₃, 17.6 mM; K₂HPO₄, 0.22 mM; MgSO₄.7H₂O, 0.03 mM; CaCl₂.2H₂O, 0.2 mM; Citric Acid.H₂O, 0.03 mM; Ammonium Ferric Citrate, 0.02 mM; Na₂EDTA.2H₂O, 0.002 mM; Na₂CO₃, 0.18 mM; H₃BO₃, 46 μM; MnCl₂.4H₂O, 9 μM; ZnSO₄.7H₂O, 0.77 μM; Na₂MoO₄.2H₂O, 1.6 μM; CuSO₄.5H₂), 0.3 μM; Co(NO₃)₂.6H₂O, 0.17 μM). The pH value of culture medium was adjusted to 7.0±0.2 before inoculation and the alga was maintained in a temperature controlled growth chamber at 25±1° C. and 100±10 μmoles/m^{2/}s light intensity provided by cool white fluorescent (6500K) T-8 bulbs.

### Example 2

Analysis of growth parameters: Chlorophyll a was estimated following the method of Porra et al., (1989) Biochem. Biophys. Acta 975, 384-394. Biomass estimations were performed by filtering the cultures through pre-dried and weighed Whatman GF/C glass fiber filters (4.7 cm diameter; 1.2 μm pore size). After washing with 0.65 M ammonium formate, filters were then dried at 60° C. overnight and cooled in a desiccant pouch before re-weighing. Specific growth rates, divisions per day, and generation times were estimated based upon biomass data for days 6 to 9 and days 9 to 12 using the following equations:

Specific growth rate (μ, d⁻¹)=ln(N₂/N₁)/(t₂−t₁)

Divisions per day (Div.day⁻¹)=μ/ln2

Generation time (d)=1/Div.day⁻¹

Where N₁ and N₂=biomass at time 1 (t₁) and time 2 (t₂), respectively.

### Example 3

Biomass Composition: Neutral lipids were estimated using an Ankom XT10 automated extractor (Chinnasamy et al., (2010) Bioresource Technol. 101: 3097-3105). Lipid content was measured gravimetrically using hexane as solvent. The same filters used for the biomass measurements (from 25 mL of culture) were used for the lipid estimation as they provided the initial weight of biomass (W₁). The filters were then placed into Ankom XT4 extraction bags, sealed with the impulse sealer and then autoclaved at 121° C. for 15 min. After drying, the extraction bags were placed in a resealable plastic bag with desiccant material while each individual bag was removed and carefully weighed (W₂). Extraction bags were then placed into the Ankom extractor and extraction was performed for 2 h at 105° C. with hexane as solvent. Bags were then transferred to a forced-air oven and dried at 60° C. overnight, then cooled in a dessicator and weighed (W₃). The following equation was used to calculate the lipid content of algal samples:

Lipid %=(W₂−W₃)/W_{1×100}

A 2% neutral lipid corn standard supplied by Ankom was used as an analytical standard. Carbon, hydrogen, nitrogen, and sulfur percentages in the biomass were estimated using a LECO CHNS932 elemental analyzer.
Chlorophyll fluorescence kinetics by PAM: The Pulse-Amplitude-Modulation Fluorometer (Mini-PAM, WALZ GmBH, Effeltrich, Germany) used for chlorophyll a fluorescence measurements were performed in real-time using the mini-PAM's internal halogen light as the actinic light source for fluorescence measurements of maximum photosynthetic yield, effective photosynthetic yield (PSII), electron transfer rate (ETR), photosynthetic quenching (qP) and non photochemical quenching (NPQ). The algal samples were dark adapted for one hour prior to the analysis. A small quantity (2.5 mL) of dark adopted sample in a 10 mm path length cuvette was placed in a dark chamber. The PAM fluorometer was connected to the sample via a fiber optic bundle. After stabilization of the auto fluorescence, the probing light beam was turned on and the baseline fluorescence (Fₒ) was recorded. A single saturating flash (1 s, 8,000 μmol m⁻² s⁻¹) was then applied to reach maximal fluorescence (Fₘ). The maximum photosynthetic efficiency of PSII was estimated as in the following equation for the dark adopted culture:

Fᵥ/Fₘ=Fₘ−Fₒ/Fₘ

The induction kinetics were initiated by turning on the actinic light source (100 μmol m⁻² s⁻¹). During the induction curve under actinic light (AL) exposure, saturating light pulses (1 s; 8,000 μmol m⁻² s⁻¹) were given every 20 seconds to determine the effective quantum yield (Φ PSII), electron transfer rates (ETR), photochemical (qP) and non-photochemical (NPQ) quenching. The qP value was determined by the PAM data analysis software, which uses Fₒ as opposed to Fₒ′ in the equation providing a estimate of qP rather than an absolute measurement using far-red light exposure. These tests were done in duplicates.

### Example 4

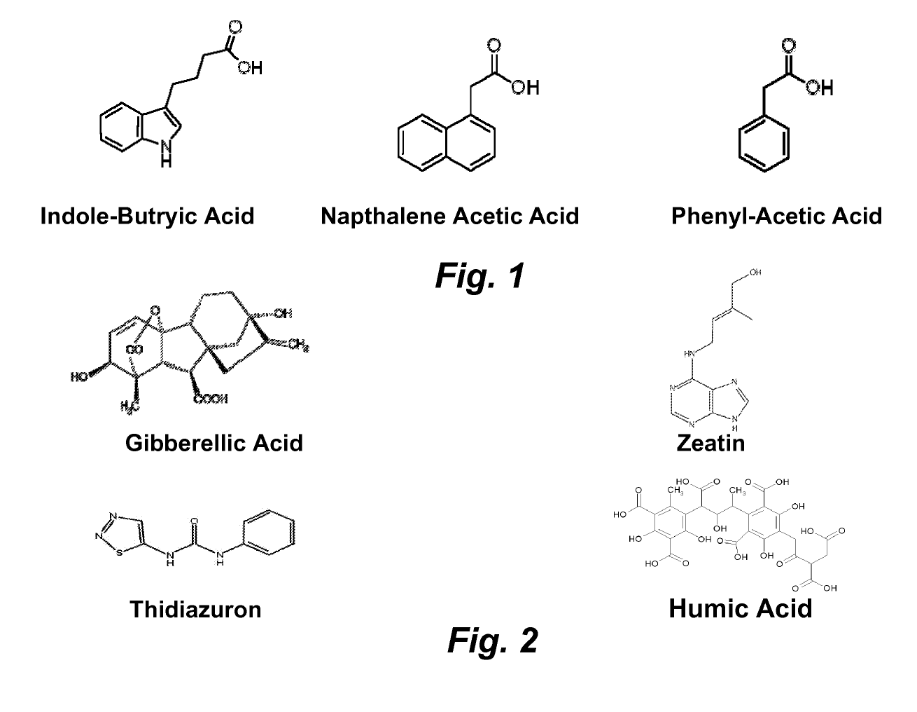

Selection of the biochemical stimulants: Ten biochemical stimulants were selected, including indole-acetic acid, naphthalene-acetic acid, and phenyl-acetic acid The structures are shown in FIGS. 1 and 2.
Biochemical stimulants: Naphthalene acetic acid (NAA), indole-butryic acid (IAA), gibberellic acid, and kelp extract were obtained from Super-Grow Plant Care, Montreal, Canada; Zeatin, and thidiazuron were obtained from bioWORLD, GeneLinx International, Inc, Ohio, USA; phenyl-acetic acid was supplied by Sigma-Aldrich, St. Louis, Mo., USA. For the purpose of screening, previously reported dosages that demonstrated growth enhancing effects were used. The details of biostimulants and their dosages are given in the Table 1.

TABLE 1
Biostimulants and their dosages used in the study

| | | Optimal | |
|---|---|---|---|
| Biostimulant | Classification | Dosage | References |
| Phenyl Acetic Acid | Auxin | 30 ppm | Brannon & Bartsch, 1939 |
| Indole-Butyric acid | Auxin | 10 ppm | Brannon & Bartsch, 1939 |
| Naphthalene Acetic Acid | Auxin | 5 ppm | Brannon & Bartsch, 1939 |
| Gibberellic Acid | Phytohormone | 10 ppm | Brian et al., 1954 |
| Zeatin | Cytokinin-like | 10 nM | Schmitz & Skoog, 1972 |
| Thidiazuron | Cytokinin | 1 μM | Thomas & Katterman, 1986 |
| Humic Acid | Growth promoter | 20 ppm | Adani et al., 1998 |
| Kelp Extract | Growth promoter | 0.025% | Crouch & van Staden, 1998 |
| Methanol | Micronutrient | 0.50% | Navakoudis et al., 2007 |
| Iron Chloride | Micronutrient | 1.2 × 10⁻⁵ M | Lu et al., 2008 |

### Example 5

The experiment devised for the testing of biostimulants was a static culture batch study performed in triplicates for all treatments. Samples were collected and analyzed on day 0, 5 and 10. The growth medium was comprised of 90 ml of BG11 with appropriate concentrations of various biostimulants. Biochemical stimulants were filter sterilized through 0.22 μm Whatman syringe filter to avoid bacterial contamination. Each flask received 10 ml inoculum of exponentially growing Chlorella sorokiniana. After inoculation, flasks were incubated in the environmental growth chamber for 10 days in a temperature controlled growth chamber at 25±1° C. and 100±10 μmoles/m²¹s light intensity provided by cool white fluorescent (6500K) T-8 bulbs.

### Example 6

Analysis: Optical density was measured at 750 nm using a UV/Visible Spectrophotometer (Varian Cary 50, Varian Inc, Palo Alto, Calif., USA). Chlorophyll content was estimated following the method of Porra et al., ((1989) Biochem. Biophys. Acta 975: 384-394, incorporated herein in its entirety). The carbohydrate and protein content were estimated using the well-known methods of Dubois and Lowry respectively. Biomass was determined by filtering 25 mL of culture using pre-weighed Whatman GF/C glass fiber filters (1.2 μm). The material was washed with deionized water, dried at 60° C. for 12 h, and weighed. Photosynthetic efficiency and kinetics were determined by chlorophyll a fluorescence analysis using a Pulse Amplitude Modulation Fluorometer (Mini-PAM Chlorophyll a Fluorometer, Heinz Walz GmbH, Effeltrich, Germany).

### Example 7

Biomass Productivity. Biomass productivity of the five and ten day old cultures was estimated. The results demonstrated increases in biomass in nearly all treatments with the exception of kelp extract which had an apparent negative impact on algal growth kinetics possibly due to the increased turbidity of the media. Naphthalene acetic acid (NAA) at 5 ppm concentration recorded a 138% increase in culture density and a cell concentration of 0.38 g/L on day 10, when compared to 0.16 g/L in the treatment with no biostimulants. However, NAA did not attain the highest growth rate during the first 5 days of growth and recorded only 47% increase in biomass production over control. The cells needed more time to acclimatize to the concentration of NAA in the medium, which could have prolonged the lag phase. Acceleration in growth rate between day 5 and 10 indicated a prolonged exponential phase which resulted in an increase in biomass productivity after the cells were adapted to the 5 ppm concentration. These results indicated the possibilities of shortening the lag phase by further reducing the concentration of NAA in growth medium without losing the biomass productivity for obtaining optimal effect.

In contrast to the above, a 95% increase in biomass during the first five days of growth was observed in a related auxin, phenyl-acetic acid (PAA) at 30 ppm dosage. The biomass productivity declined after five days and it was only 42% more when compared to the treatment without the biostimulant on day 10, indicating a role for PAA in shortening initial lag period.

The third auxin used in the experiment, indole-butryic acid (IBA) at 10 ppm, recorded a 74 and 76% increase in biomass production over control for day 5 and 10, respectively. IBA demonstrated a balance between the two other auxins, NAA and PAA. The IBA treatment reached a biomass productivity of 0.28 g/L which was approximately the same as gibberellic acid (GAA) on day 10. However GAA barely surpassed the control on day 5 with some substantial variance. The much more complex chemical composition of humic acid demonstrated a 43% increase over control at 20 ppm concentration, which gave a light brown tinge to the media. The non-auxin phytohormones, such as the cytokinin: zeatin (ZT) and cytokinin-like: thidiazuron (TDZ), demonstrated 27% and 33% growth over control. The weaker response to these two treatments could be due to the extremely low dosage or uneven dissolution during preparation rendering zeatin not as effective as the other phytohormones. The only mineral nutrient tested was ferric chloride, which exhibited only marginal increases and decreases on day 10 and 5 with some variation. However, such large doses are used for enhancing metabolite synthesis as opposed to biomass growth.

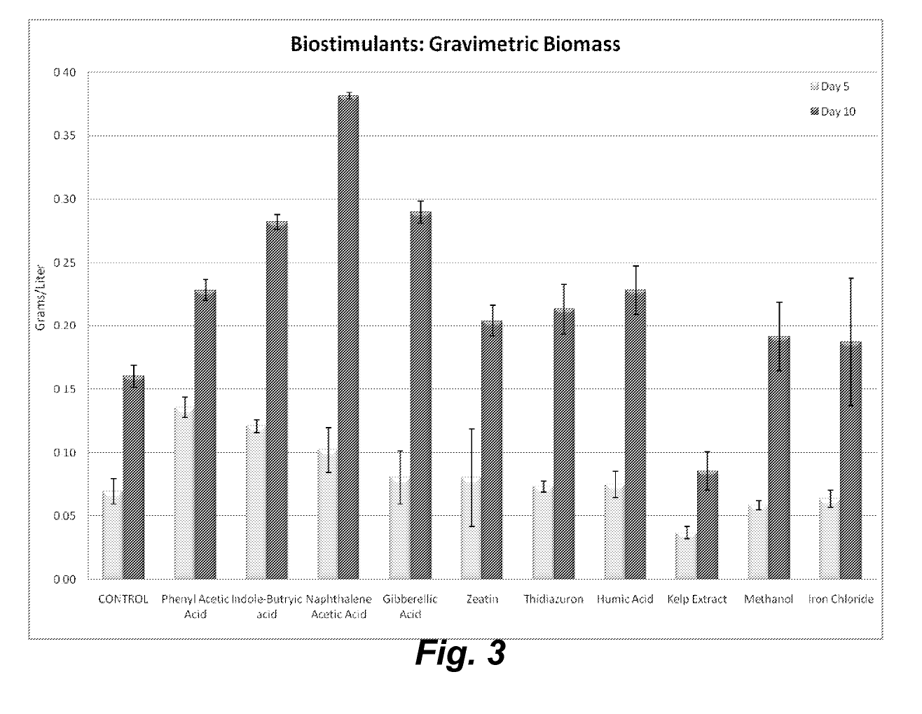

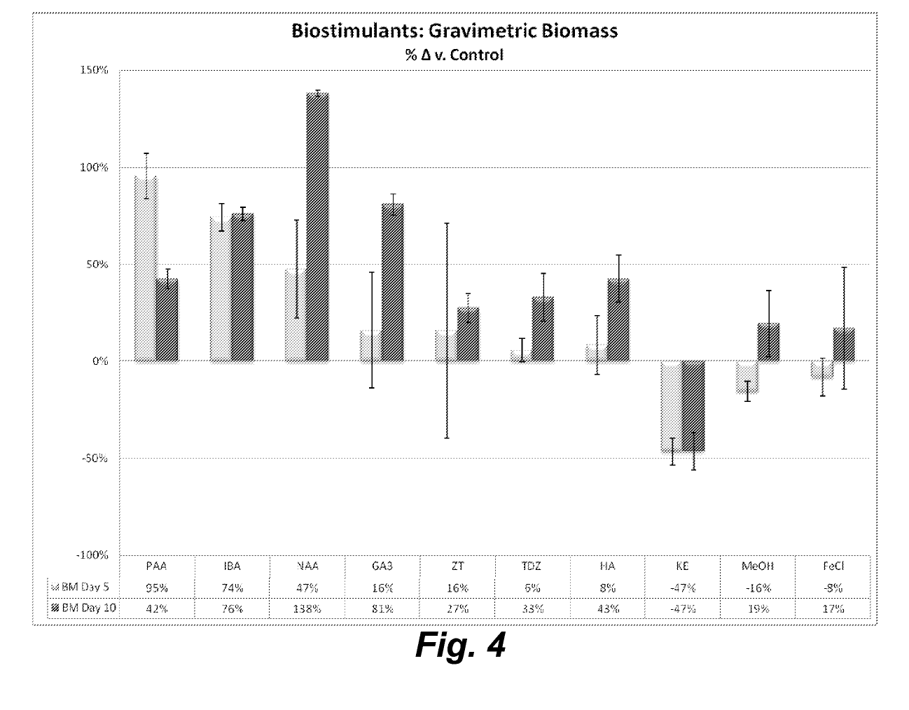

Some of the large variations observed in the ferric chloride and zeatin treatments, which could be due to uneven dissolution of zeatin in solution as well as some degree of bacterial contamination. From the biomass data, the auxins such as NAA, PAA and IBA were most effective for enhancing growth, despite the variation found in optimal stimulation or induction time for the test alga (as shown in FIGS. 3 and 4).

### Example 8

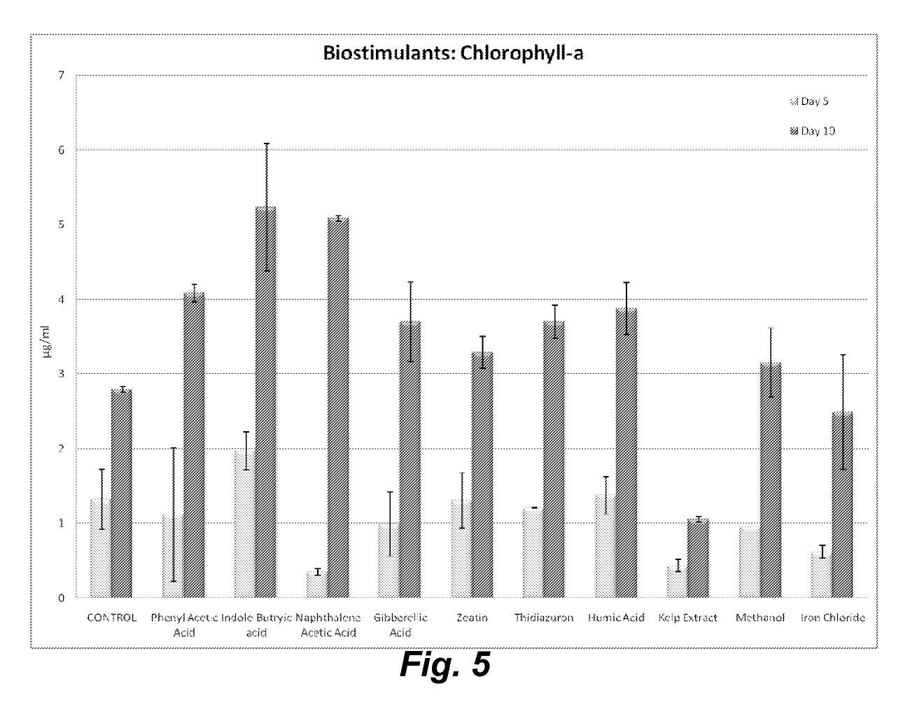

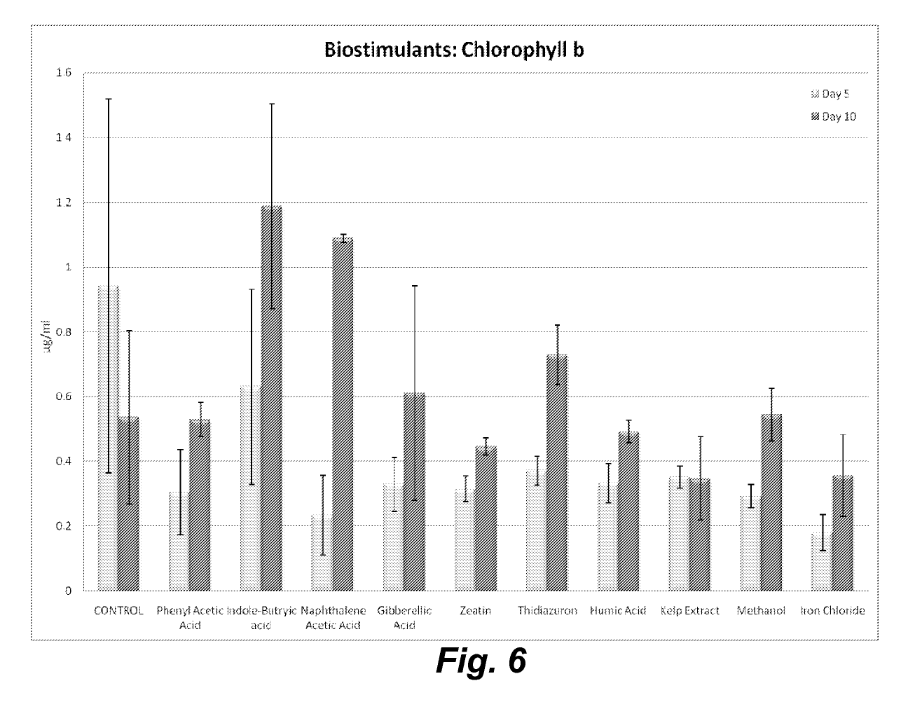

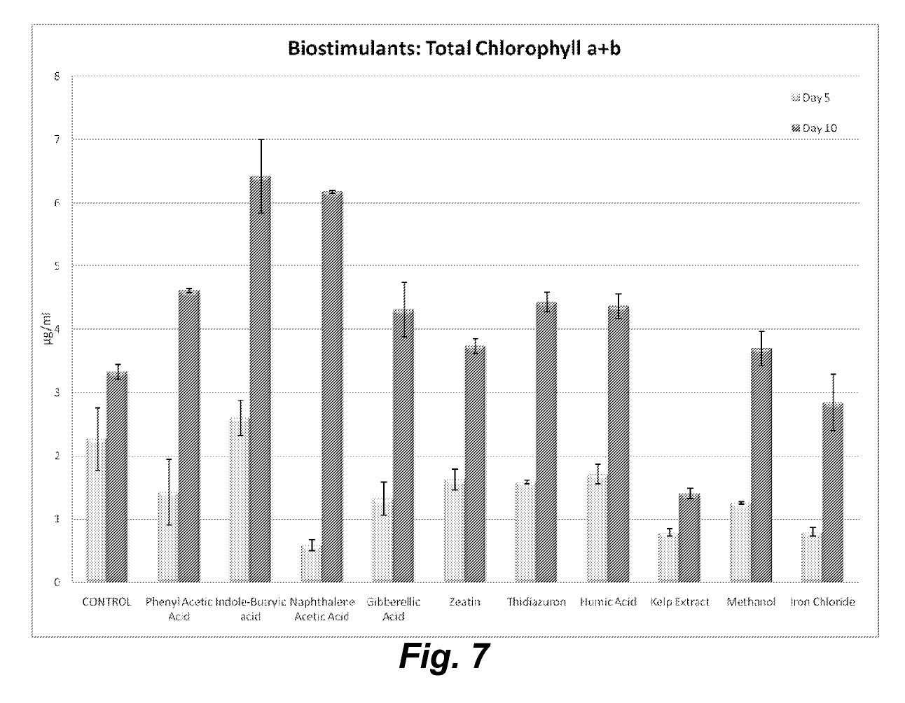

Chlorophyll analysis: The results showed substantial increases in chlorophyll a (FIG. 5), chlorophyll b (FIG. 6), and total chlorophyll (FIG. 7) particularly for the auxin group on the final sampling day 10. The highest increase was exhibited by IBA by day10, attaining a 93% increase over control, but only had a marginal increase by day 5. NAA reached a similar chlorophyll productivity on day 10, which showed an increase of about 85% over control. The IBA treatment was the only one that showed any potential increase in chlorophyll on day 5. All the remaining treatments showed no significant increase in chlorophyll content on day 5. Suprisingly the strongest inhibition was found in NAA showing a 74% decrease, followed by 65, 45 and 37% decrease in ferric chloride, MeOH and PAA, respectively.

PAA and NAA were two of the highest performers for biomass concentration for day 5 and day 10, respectively. NAA showed 74% decrease in chlorophyll content on day 5 and recorded 85% increase on day 10, indicating a substantial increase in growth rate between day 5 and 10. The kelp extract treatment performed poorly demonstrating a 65% and 58% decrease over control for day 5,10 respectively. The treatments containing GA, TDZ and HA demonstrated a moderate increase of 30% in chlorophyll content of over control, which corresponds to approximately 30% increase in biomass on day 10. Chlorophyll b content seemed to have larger deviations when compared to chlorophyll a, however, these quantities are low, and constitute a small fraction in the total chlorophyll.

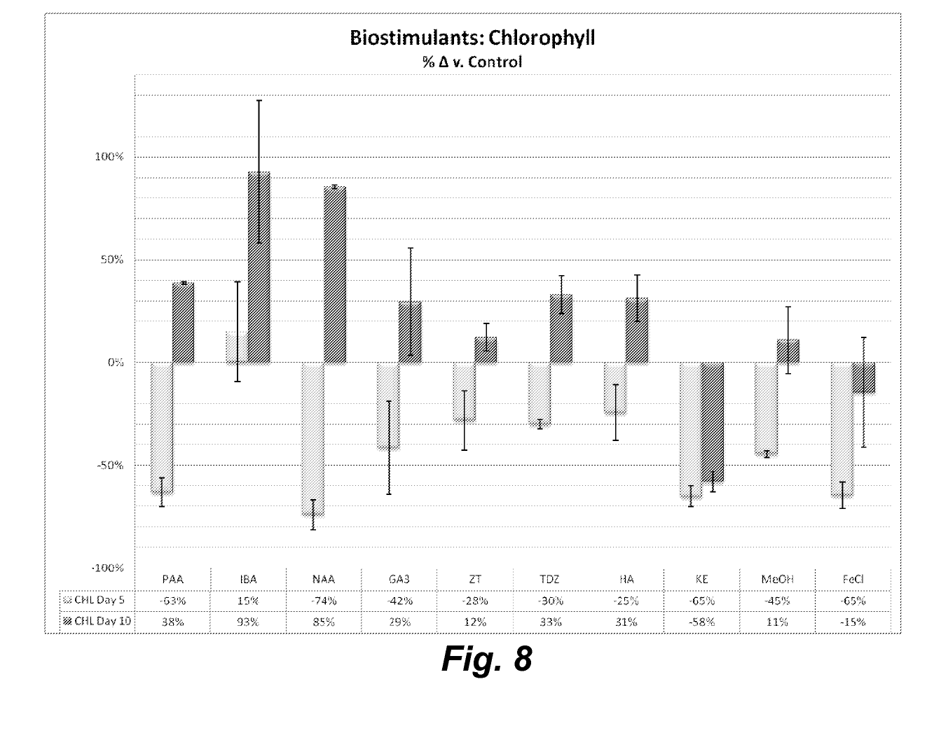

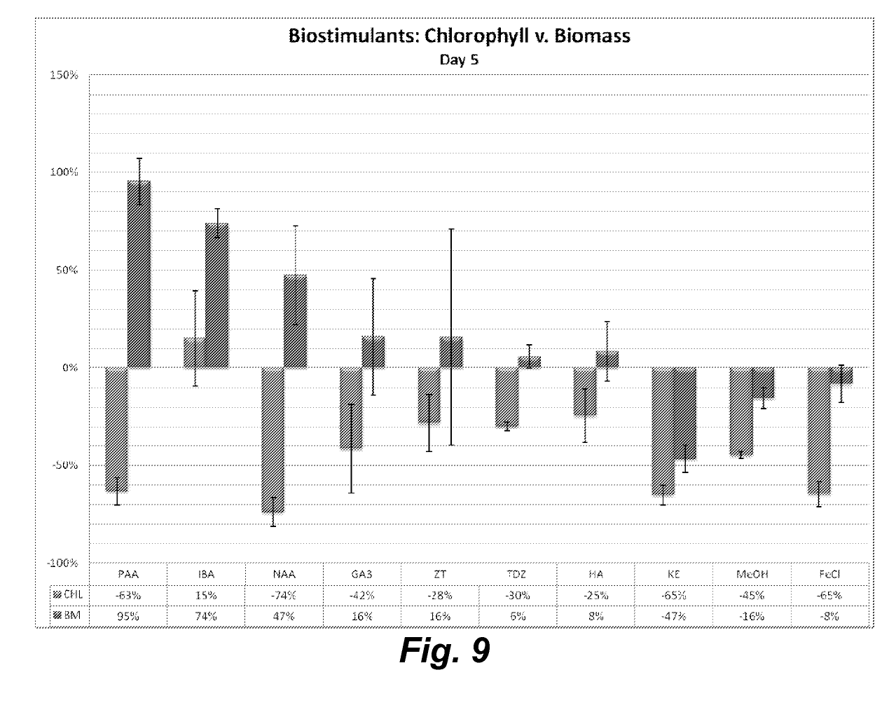

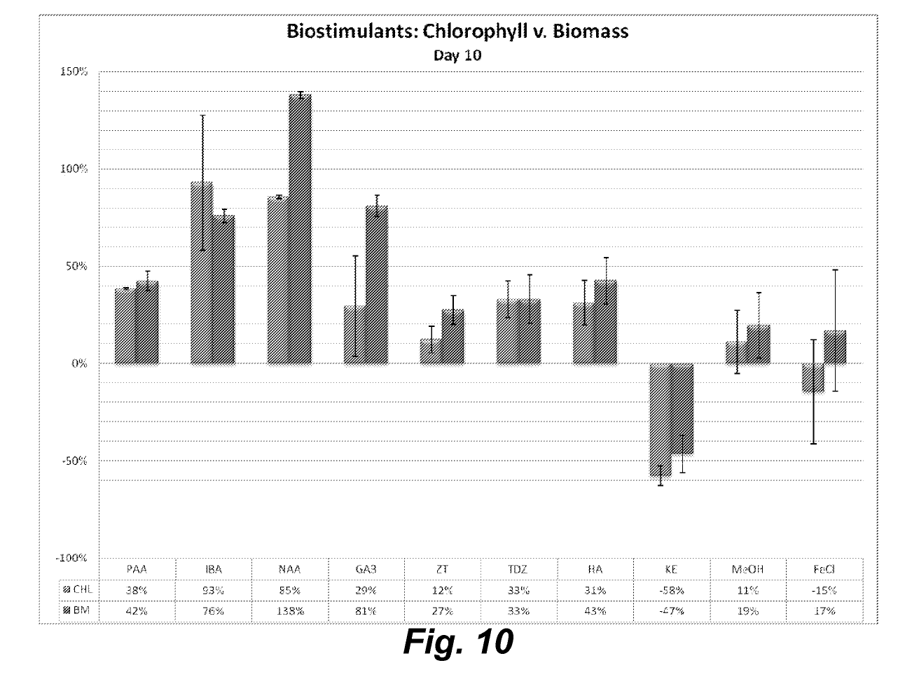

Upon examining the comparison of changes in chlorophyll and biomass versus control (FIG. 8), the auxins demonstrated an interesting phenomenon on day 5. PAA, IBA and NAA all had reduced chlorophyll synthesis and in the case of IBA, marginal changes in chlorophyll synthesis, while simultaneously recording substantially higher biomass concentrations (95%, 74% and 47%, respectively, as shown in FIGS. 9 and 10). IBA seems to preferentially increase chlorophyll synthesis over biomass and other metabolites. This phenomenon is attractive because in chemical processing and fractionation of algal biomass, chlorophyll pigments interfere with extraction procedures causing problems downstream. With these auxin treatments there apparently exists some mechanism that can reduce pigment production while promoting massive increases in biomass productivity.

### Example 9

Carbohydrate Analysis: Auxins recorded the highest carbohydrate content compared to the other growth promoting substances, as shown in FIG. 11. IBA and NAA exhibited a significant increase in carbohydrate concentration by day 10 which was between about 87 to about 92% over control. The stimulation of IBA seemed to maintain a close relationship between the enhanced carbohydrate content and the biomass increase suggesting a general stimulation of total growth. PAA demonstrated a smaller enhancement of 27% over control, but was also accompanied with substantial deviation rendering marginal enhancement at best. Zeatin recorded an increase of 31% in carbohydrate content on day 10, while this treatment was recorded reduced carbohydrates synthesis on day 5. In fact, all treatments showed an inhibitory effect on carbohydrate synthesis on day 5 versus control. IBA, NAA and PAA all had reduced carbohydrate contents on day 5, while simultaneously inducing large increases in biomass signifying that the increase in biomass was due to other metabolites such as proteins or lipids.

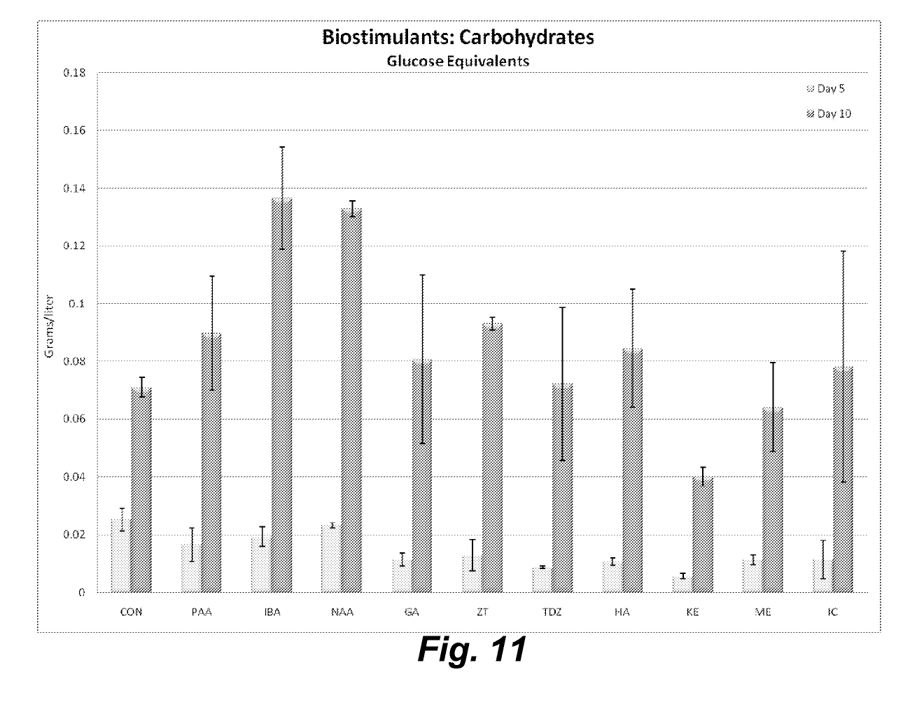

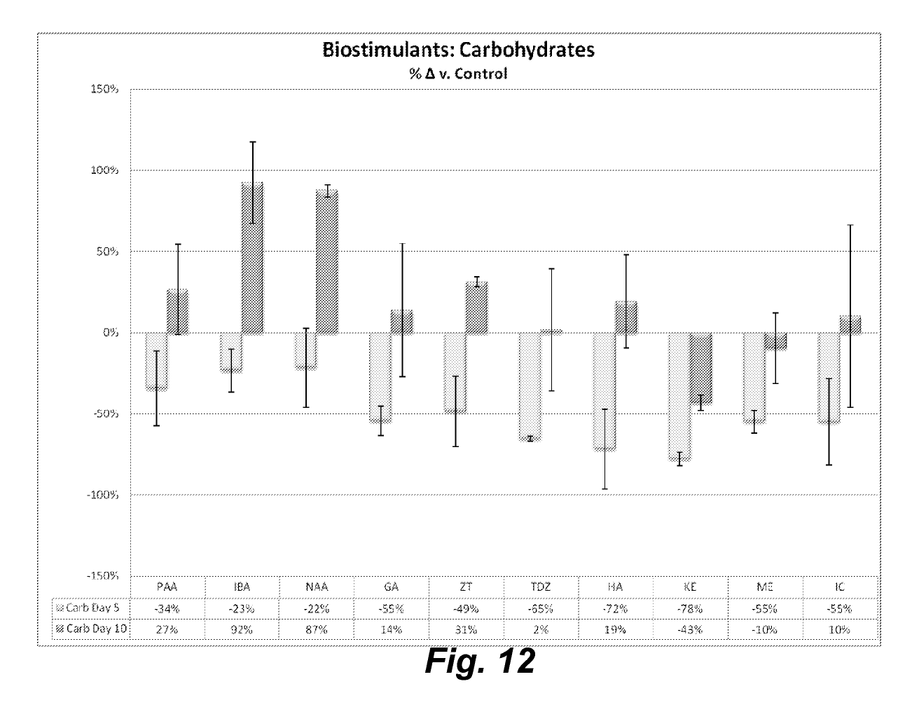

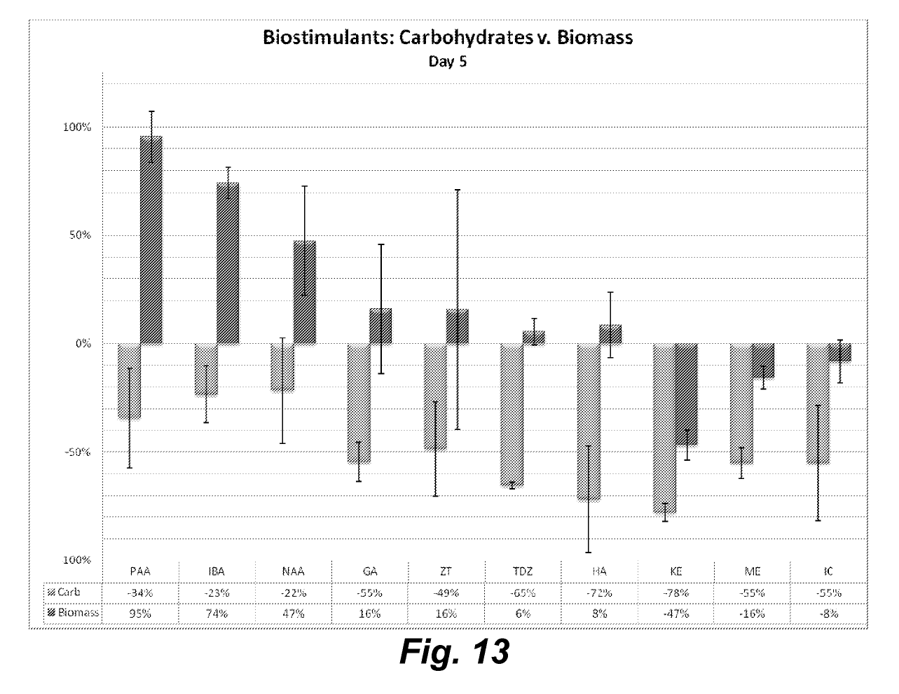

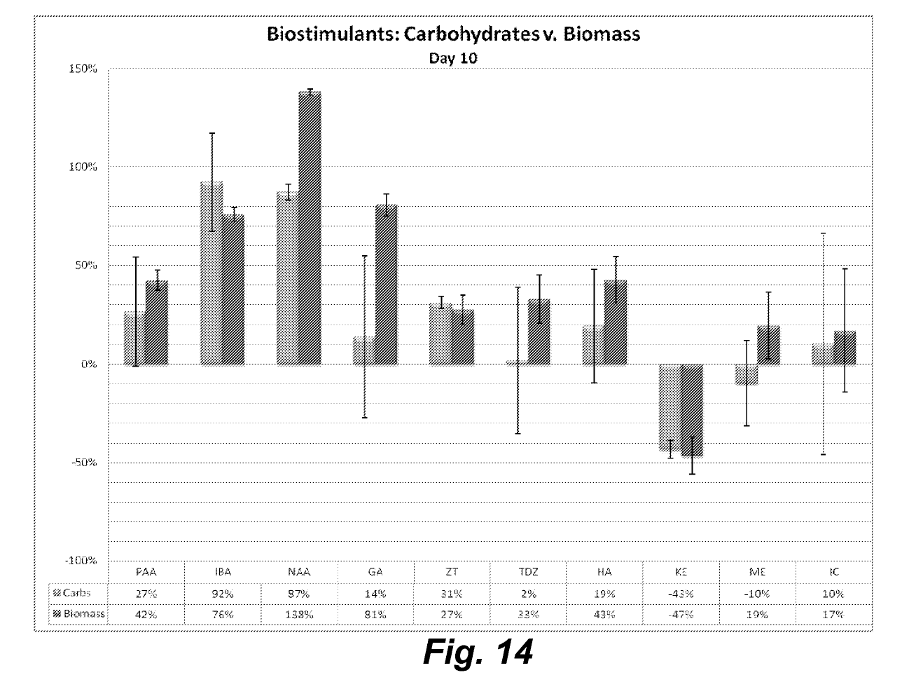

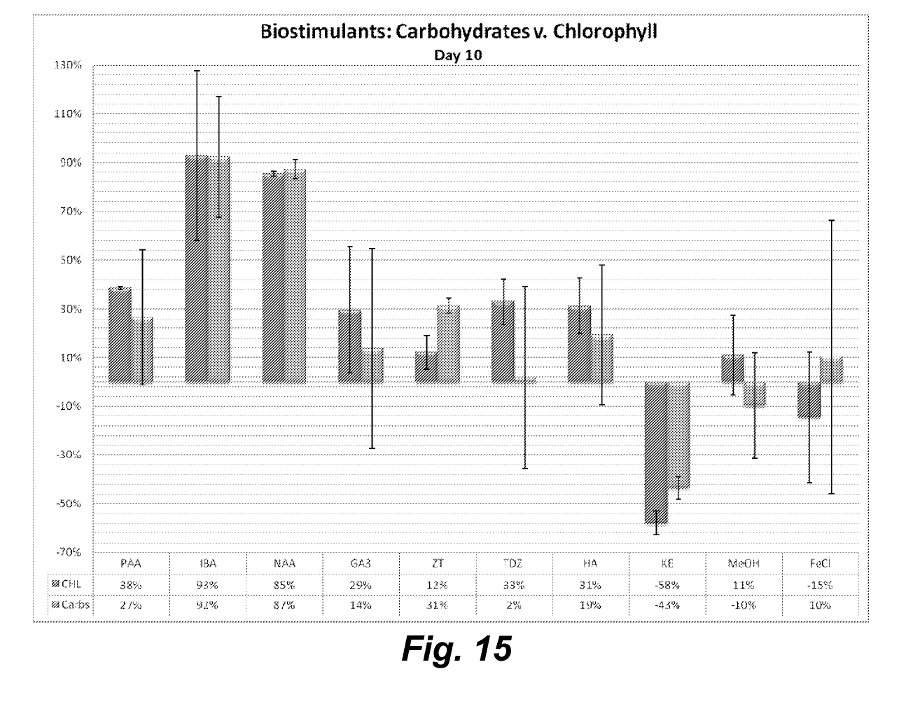

Humic acid showed significant decrease in carbohydrate synthesis (about 72%) compared to 8% increase in biomass during the same time interval. This indicates the possibility of diversion of photosynthetically fixed carbon for the synthesis of other metabolites such as proteins and lipids. The relationship between chlorophyll and carbohydrate synthesis seems to be more closely related with respect to the auxins. In the case of IBA and NAA, the chlorophyll and carbohydrate increases verse control mirror each other showing a tight correlation between two parameters, where as there is a larger comparative increase in biomass in the case of NAA (as shown FIGS. 11 to 15).

### Example 10

Test strain: Chlorella sorokiniana (UTEX 2805) was obtained from UTEX Culture Collections and maintained in BG11 medium (Stanier et al., (1971) Bacteria Rev. 35: 171-205, incorporated herein in its entirety). The pH of culture medium was adjusted to 7.5±0.2 before inoculation and the alga was maintained in a temperature controlled growth chamber at 25±1° C. and 100±10 μmol m⁻² s⁻¹ light intensity with 12/12 h L/D cycle provided by cool white fluorescent (6500 K) T-8 bulbs.
Pesticide: A twelve day time-scale growth study was conducted to evaluate the effect of pesticide toxicity on algal growth and metabolism. The pesticide malathion (Spectracide, 50% v/v) was used and added to fresh BG11 medium to obtain final concentrations of 70, 140, 280 and 560 mg of malathion L⁻¹ for the treatments. Control sets did not include the pesticide. After sterilization, C. sorokiniana was added as inoculum to obtain an initial biomass concentration of 0.075 g L⁻¹ in all the treatments in triplicates. The cultures were incubated in the growth chamber as explained above. For each sampling period, three randomly chosen flasks of each treatment were removed and then refrigerated at 4° C. until further analysis.

### Example 11

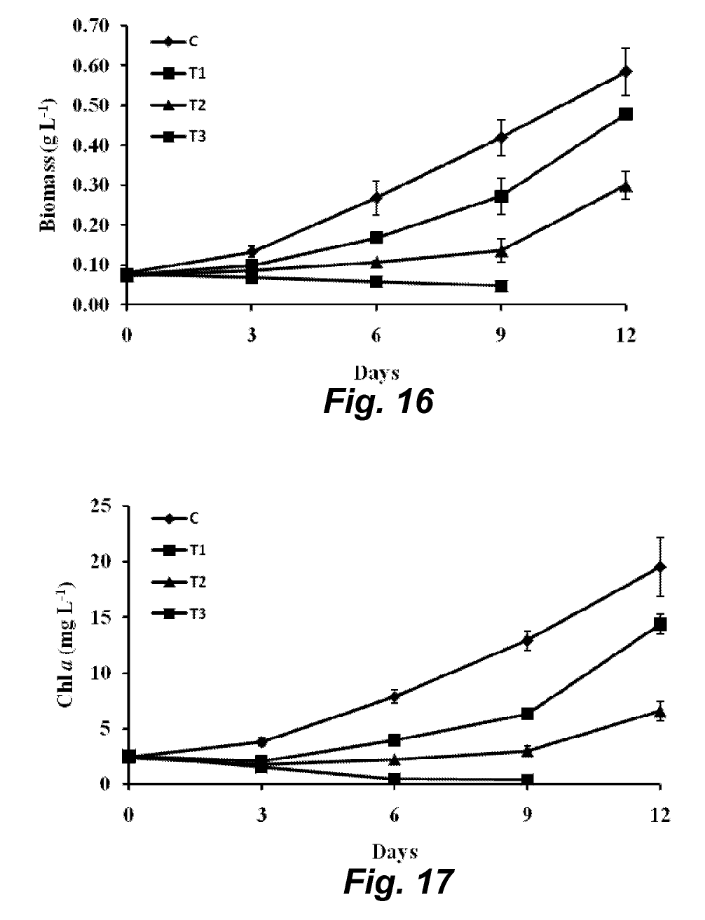

Biomass and Chlorophyll: Over the 12 days growth, biomass and chlorophyll production showed similar trends (FIGS. 16 and 17). The treatments with 70 mg malathion L⁻¹ showed 25%, 37%, 35% and 18% inhibition in biomass and 45%, 49%, 51% and 26% inhibition in chl a on day 3, 6, 9 and 12, respectively. The treatments with 140 and 280 mg of malathion L⁻¹ showed 35% and 48%, 60% and 78%, 68% and 89% and 49 and 100% inhibition of biomass on day 3, 6, 9 and 12, respectively.

Over the 9 day period it was monitored, the treatments with 280 mg malathion L⁻¹ continuously lost biomass, and thus chlorophyll a. The treatments with 70 and 140 mg malathion L⁻¹, however, recovered from inhibition after day 6 and 9, respectively as per the biomass data; whereas chl a showed the recovery after day 9 in both the treatments (FIGS. 16 and 17). Between day 9 and 12, the treatments with 70 and 140 mg malathion L⁻¹ exhibited most pronounced biomass and chlorophyll a increases compared to the steady increase observed in the control from day 3. Even on day 12, the treatments with 70 and 140 mg malathion L⁻¹ showed exponential growth of algae as the cells would have recovered from the inhibition caused by the pesticide. Organophosphorus insecticides like malathion have been reported to inhibit plant cytochrome P450 monooxygenases in terrestrial plants (Biediger et al., (1992) Weed Technol. 6: 807-812; Kapusta & Krausz (1992) Weed Technol. 6: 999-1003). Cytochrome P450 monooxygenases play a key role in detoxifying xenobiotics and degrading pesticides in plants and algae (Munkegaard et al., (2008) Ecotoxicology 17: 29-35). Thies et al. ((1996) Plant Physiol. 112: 361-370) reported that the green alga Chlorella fusca had a wide range of P450 enzymes for pesticide degradation. Hence the recovery of algal cells from inhibition caused by 70 and 140 mg malathion L⁻¹, could be due to the pesticide degradation facilitated by cytochrome P450 monooxygenases.

Christie ((1969) Water Sewage Works 116: 172-176) reported that 100 mg of malathion L⁻¹ had very little effect on the growth of green alga Chlorella pyrenoidosa. However, malathion had a partial inhibitory effect on the growth of the blue green alga Chlorogloea fritschii and it permanently suppressed the growth at 200 mg L⁻¹ (Lal & Lal, (1988) Pesticides and Nitrogen Cycle, Vol 3. CRC, Boca Raton, Fla., USA). Malathion inhibited chlorophyll production in Stigeoclonium, Tribonema, and Vaucheria by 100% at a concentration of 1 μg L⁻¹ was reported (Torres & O'Flaherty (1976) Phycologia 15: 25-36). They also proved that algae can degrade malathion in the presence of light. Tiwari et al., ((1979) J. Sci. Res. 30: 92-96) reported that the growth of Nostoc calcicola was reduced by malathion only above 500 mg L⁻¹. However, Munkegaard et al., ((2008) Ecotoxicology 17: 29-35) found that malathion when applied alone at concentrations equal to half their solubility in water (75 mg L⁻¹) was toxic to algae.

Anabaena was found to be highly resistant to malathion even at 500 mg L⁻¹ without any bleaching (Tandon et al., 1988 Environmental Pollution 52: 1-9). It was also found that the inhibition was maximum during lag phase and was reduced appreciably when the alga entered into exponential growth phase. The inhibition caused by 500 mg malathion L⁻¹ was reduced from 64% on day 5 to 15% on day 30.

Murray & Guthrie ((1980) Bull. Environ. Contain. Toxicol. 18: 525-542) observed that organophosphorus insecticides appear to inhibit algal growth initially, but the inhibition is usually short lived, with the algae eventually returning to control levels. The present findings were in accordance with this observation, and malathion appears to be safe to the alga when used at recommended levels.

Pesticide induced inhibition of photosynthesis due to prevention of chloroplast electron flow through photosystem II was earlier reported in Chlorella protothecoides (Subbaraj & Bose (1983) Biochem. Physiol. 20: 188-193; Singh & Vaishampayan (1978) Environ. Expt. Bot. 18: 87-94). It is evident from these studies that malathion impacts energy production via photosynthesis and disrupts synthesis of protein subunits necessary for photosynthesis by blocking electron transfer in the photosynthetic process.

In the present study, biomass and chlorophyll a for the treatments with 560 mg malathion L⁻¹ could not be estimated due to the lethal effects of malathion.

### Example 12

Chl a/b ratio: The control and the treatments with 70 and 140 mg malathion L⁻¹ displayed a near uniform ratio of chl a to chl b beginning at approximately 0.85 on day 0 and ending at approximately 0.90 on day 12. The 280 mg malathion L⁻¹, though, caused approximately 100% decrease of chl a/b ratio. This clearly indicates the inhibitory effect of malathion on chlorophyll a synthesis which is in confirmation with the earlier reports on pesticide toxicity on algae (Kaushik & Venkataraman (1993) Curr. Sci. 52: 321-323; Kumar et al., (2008) Science of the Total Environment 403: 130-138).

### Example 13

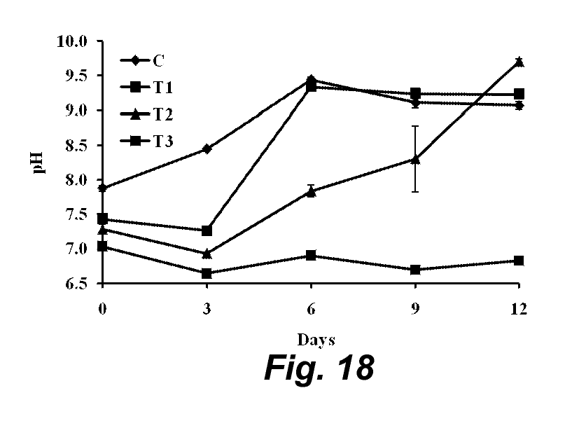

pH: Each malathion treatment exhibited an initial drop in pH on day 3 (FIG. 18), possibly due to the inhibition of photosynthetic activity caused by the pesticide. However, the treatments with 70 and 140 mg malathion L⁻¹ recovered from this inhibition and showed an increase in pH between day 3 and 6. By day 6, the control and the treatment with 70 mg malathion L⁻¹ reached a maximum pH value of about 9.5 and trended to about 9.0 on day 12. In contrast, the 140 mg malathion L⁻¹ exhibited a near linear pH increase up to 9.6 on day 12. But the treatment with 280 mg malathion L⁻¹ recorded low pH values below 7 on day 3, 6, 9 and 12 due to a reduction in the photosynthetic activity.

### Example 14

Specific growth rate: Day 9 to 12 biomass data apparently showed simultaneous exponential growth among control and the treatments with 70 and 140 mg malathion L⁻¹. Between day 9 and day 12, the treatment with 140 mg malathion L⁻¹ showed 2.2 times increase in biomass production, highest growth rate (0.26 d⁻¹) and shortest generation time (2.6 d) when compared to the control and the treatment with 70 mg malathion L⁻¹ (Table 2).

TABLE 2
Growth responses of Chlorella sorokiniana in different
concentrations of malathion between day 6 and 9 and
day 9 and 12.

| | | | Increase in | Specific | | |
|---|---|---|---|---|---|---|
| | | Δ | biomass | growth | | Gener- |
| Treat- | | Biomass | production | rate | Divisions | ation |
| ment | Day | (g L⁻¹) | (times) | (μ) | per day | time |
| Control | 6 to 9 | 0.151 | 1.56 | 0.15 | 0.21 | 4.7 |
| | 9 to 12 | 0.165 | 1.39 | 0.11 | 0.16 | 6.3 |
| T1 | 6 to 9 | 0.103 | 1.61 | 0.16 | 0.23 | 4.4 |
| | 9 to 12 | 0.206 | 1.76 | 0.19 | 0.27 | 3.7 |
| T2 | 6 to 9 | 0.029 | 1.27 | 0.08 | 0.12 | 8.7 |
| | 9 to 12 | 0.163 | 2.20 | 0.26 | 0.38 | 2.6 |

C-Control (BG11 only); T1-70 mg of malathion L⁻¹; T2-140 mg of malathion L⁻¹; Results indicated that there was no growth observed in the treatments T3 and T4 (560 mg of malathion L⁻¹). Both the treatments proved lethal. Values are means±S.D with n=3.

However, the increase in biomass between day 9 and 12 was almost comparable for control (0.165 g L⁻¹) and 140 mg malathion L⁻¹ (0.163 g L⁻¹), whereas the treatment with 70 mg malathion L⁻¹ gained during the last three days. Generation times for day 9 to 12 in the treatments with 70 and 140 mg malathion L⁻¹ showed an interesting trend as it was 3.7 and 2.6 days, respectively when compared to 6.3 days in the control (Table 2). These results indicate that malathion inhibited algal cells in the early stages of growth and prolonged the lag period. However, the cells after adaptation, showed exponential growth which is evident from the results. These results also established that the inhibition was a dose-dependent response. However, malathion in higher concentrations viz. 280 and 560 mg L⁻¹ proved lethal as there were no signs of recovery even on day 12. Corresponding generation times for the treatment with 280 mg malathion L⁻¹ could not be calculated since these cultures did not show any growth and experienced strong inhibition by malathion.

### Example 15

Carbon, nitrogen, hydrogen, sulfur and proteins: Carbon, hydrogen, nitrogen and sulfur content of the algal biomass harvested on day 12 from the treatments with 0, 70, and 140 mg malathion L⁻¹ is presented in Table 3.

TABLE 3
Biomass composition of 12 day old culture of Chlorella sorokiniana
grown in different concentrations of malathion.

| | | | | | | | | | | | Protein | |
|---|---|---|---|---|---|---|---|---|---|---|---|---|
| | C (%) | | H (%) | | N (%) | | S (%) | | C/N ratio | | (%) | |
| Treatment | Mean | SD | Mean | SD | Mean | SD | Mean | SD | Mean | SD | Mean | SD |
| C | 46.6 | 0.2 | 7.8 | 0.03 | 9.2 | 0.2 | 0.6 | 0.1 | 5.06 | 0.11 | 57.7 | 1.4 |
| T1 | 44.8 | 0.7 | 7.4 | 0.1 | 9.2 | 0.1 | 0.6 | 0.03 | 4.86 | 0.04 | 57.6 | 0.8 |
| T2 | 45.0 | 2.2 | 7.8 | 0.6 | 9.6 | 0.8 | 0.6 | 0.02 | 4.69 | 0.17 | 60.1 | 4.7 |

C-Control (BG11 only); T1-70 mg of malathion L⁻¹; T2-140 mg of malathion L⁻¹; Results indicated that there was no growth observed in the treatments T3 and T4 (560 mg of malathion L⁻¹). Both the treatments proved lethal. Values are means±S.D with n=3.

The carbon percentages of the three treatments were nearly identical as were the nitrogen percentages. The resulting C/N ratios were narrow and not showed any significant difference (5.06±0.11, 4.86±0.04, 4.69±0.17 for 0, 70 and 140 mg malathion L⁻¹, respectively). Day 12 biomass nitrogen percentages point to a near uniform protein content among the three treatments of about 57-60%. It is evident from the results that the algal cells could overcome the inhibition caused by the malathion after day 9 through the synthesis of enzymes involved in degradation of xenobiotic compounds. The data also confirms that the photosynthetic electron flow was restored in the treatments with pesticides as the carbon and protein content in the treatments were comparable with control.

### Example 16

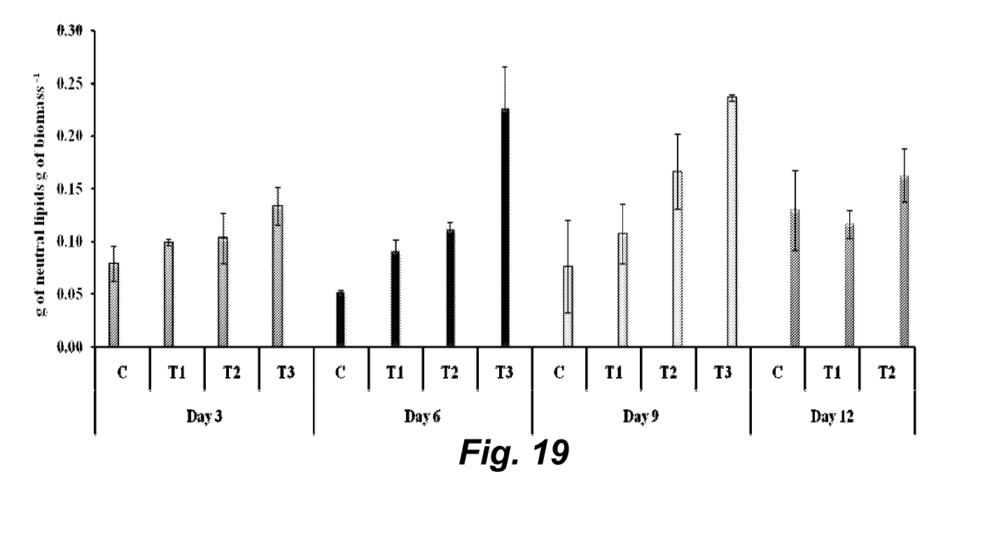

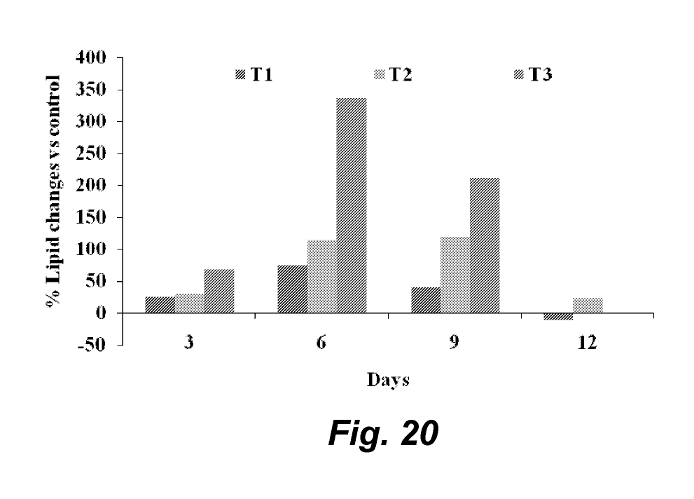

Lipids: Neutral lipid productivity mirrored the biomass and chlorophyll a productivity trend. The mean neutral lipid productivity values closely match the biomass and chlorophyll trends over the course of the 12 days. However, the changes in the lipid content in the cells showed an enhancing trend on day 3, 6 and 9 for the treatments with 70, 140 and 280 mg of malathion L⁻¹ (FIG. 19). On day 6, treatments with 70, 140 and 280 mg malathion L⁻¹ resulted in 70, 110 and 325% increase in lipid production over the control (FIG. 20).

### Example 17

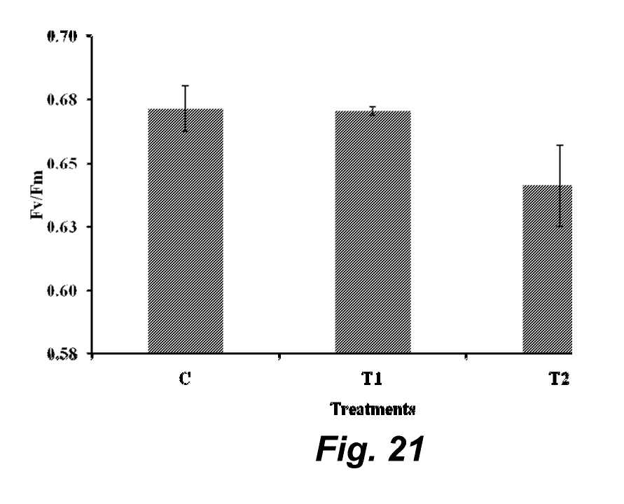

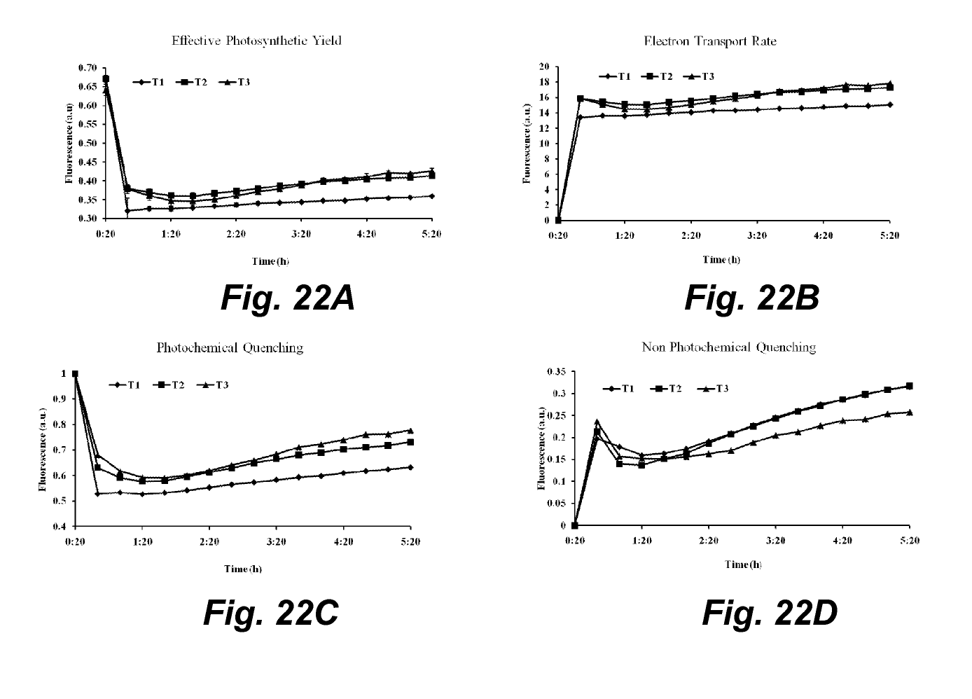

Chlorophyll fluorescence kinetics: Day 12 pulsed amplitude modulation fluorometry of dark-adapted samples showed no major discrepancy for the maximum photosynthetic efficiency among the three treatments sampled (FIG. 21). The Fᵥ/Fₘ values showed no significant difference for the control (0.672) and the treatment with 70 mg malathion L⁻¹ (0.671), whereas the treatment with 140 mg malathion L⁻¹ showed a slight decrease (0.641). The effective photosynthetic yield, electron transfer rate and photochemical quenching were about 1.2 times higher in the treatments with 70 and 140 mg malathion L⁻¹ over the control (FIG. 22A-22B).

Non-photochemical quenching (NPQ) in the treatment with 140 mg malathion L⁻¹ was 18% less than the control; whereas the NPQ of treatment with 70 mg malathion L⁻¹ was comparable with the control (FIG. 22D). Pulse-amplitude-modulated (PAM) fluorometer was used in toxicity investigations earlier to study photosynthesis, as chlorophyll fluorescence kinetics of different fluorescence parameters provide reliable information of the effect of abiotic and biotic stresses on plant physiology.

The most frequently used parameters are effective photosynthetic yield, electron transfer rate, photochemical quenching and NPQ (Juneau et al., (2003) Arch. Environ. Contam. Toxicol. 42: 155-164). On day 12, the treatment with 140 mg malathion L⁻¹ showed better performance than the control and 70 mg malathion L⁻¹, with respect to effective photosynthetic yield, electron transfer rate, photochemical quenching and non-photochemical quenching. Chlorophyll fluorescence parameters clearly indicate that the alga could overcome malathion induced stress by metabolizing it and resuming normal photosynthetic and metabolic activities after day 6 for the treatment with 70 mg malathion L⁻¹ and day 9 for the treatment with 140 mg malathion L⁻¹. Due to nutrient depletion the photosynthetic activity and growth rate of alga declined in control between days 9 and 12, which was evident from the data on chlorophyll fluorescence kinetics and specific growth rate of 0.11, whereas the treatments with 70 and 140 mg malathion L⁻¹ showed 1.8 and 2.2 times increase in biomass production and a specific growth rate of 0.19 and 0.26, respectively.

NPQ was comparatively less in the treatment with 140 mg malathion L⁻¹ which could be due to the presence of carotenoids produced in the alga acting as electron sinks to counteract pesticide toxicity. Also there was no significant difference in the carbon and protein contents of the alga in control and treatments with 70 and 140 mg malathion L⁻¹. The pH of the 12 day old culture of the treatments with 70 and 140 mg malathion L⁻¹ were 9.71 and 9.23, respectively whereas it was 9.07 for the control. It further confirms that the photosynthetic activity of malathion treated alga (70 and 140 mg malathion L⁻¹) was no longer inhibited by pesticide toxicity. However, the treatments with 280 and 560 mg malathion L⁻¹ proved lethal and the algal cells never recovered from the stress. This study indicates that the alga can withstand malthion concentration up to 140 mg L⁻¹.

Currently there is greater interest in identifying oleaginous algal strains which produce more lipids for production of biofuels. However, it has been widely reported that growth and lipid production in algae are mutually exclusive as lipid accumulation occurs only during the stationary phase. Hence, induction of lipids in non-oleaginous algae without compromising with growth will be highly beneficial for commercial cultivation of algae to produce biofuels.

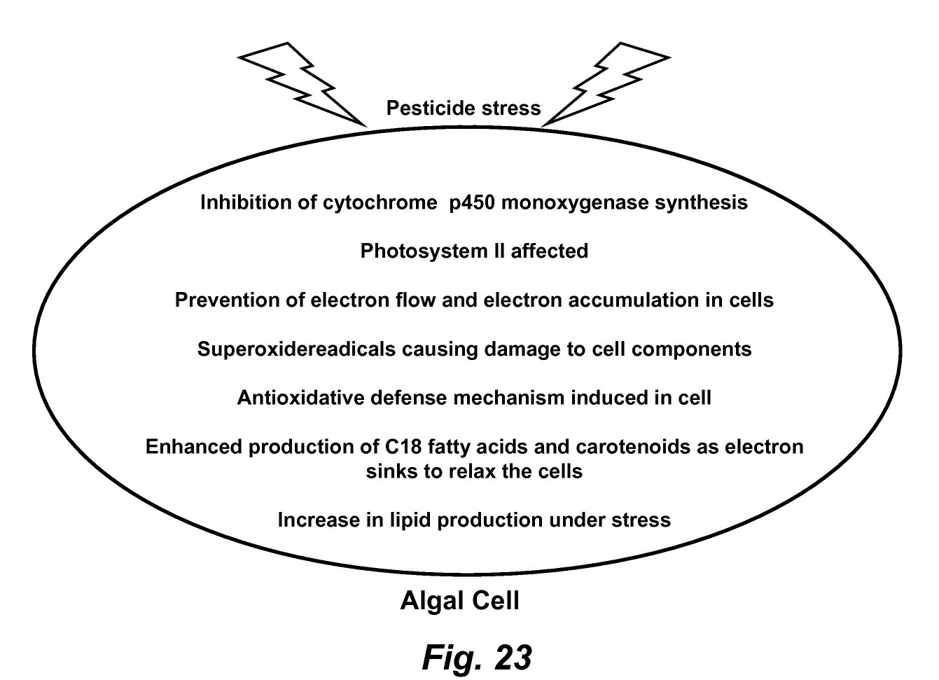

Nitrogen starvation was reported to induce the production of triglycerides (TAG) in algae (Rodolfi et al., (2009) Biotechnol. Bioeng. 102: 100-112)). Wang et al. ((2009) Eukaryotic Cell 8: 1856-1868) observed that lipid accumulation fails to occur without N-starvation, indicating the existence of a nitrogen trigger or, a stress trigger. In the present study it was observed that the pesticide stress caused significant increases in lipid accumulation over control which is in confirmation with the earlier findings on stress related lipid production. The possible mechanisms involved in the pesticide mediated growth responses and lipid induction are depicted in FIG. 23.

The alga tolerated malathion concentration up to 140 mg L⁻¹. The alga could metabolize the pesticide and overcome the stress within 6 days in the treatment with 70 mg malathion L⁻¹ and 9 days with 140 mg malathion L⁻¹. Chlorophyll fluorescence studies of the alga also confirmed the reversal of pesticide inhibition in these treatments. However, the treatments with 270 and 540 mg malathion L⁻¹ proved lethal.

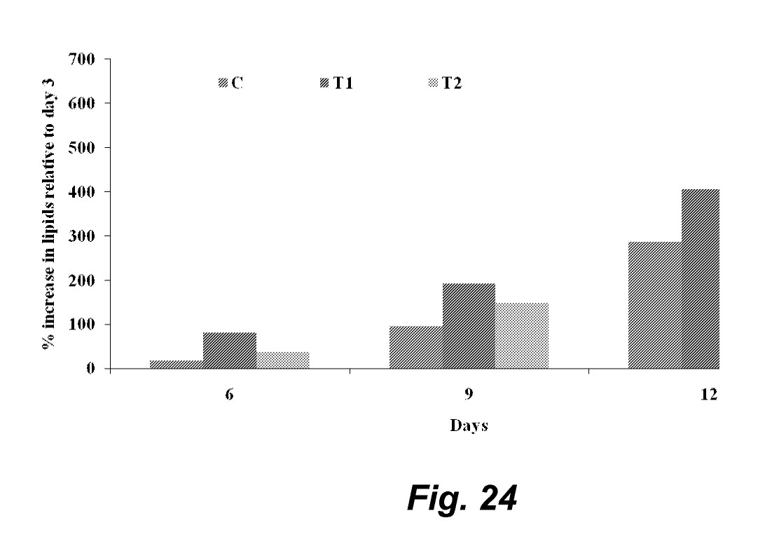

This study indicates the use of pesticides like malathion for inducing lipids in algae. The experiments revealed that 70-325% increase in lipid production can be achieved using stress stimulants like malathion (a pesticide) at concentrations ranging from 70-280 mg L⁻¹ when used in the growth medium. The projected increase in the lipid content of the cells based on the experimental results observed in this study is given in Table 4 and FIG. 24.

TABLE 4
Projected increase in the lipid content of the cells based on the
experimental results observed in this study and considering the
initial density of the cells as constant in control and other treatments.
malathion (280 mg L⁻¹) as stress stimulant.

| | | | Changes in | | | |
|---|---|---|---|---|---|---|
| | | | lipid content | | Day 12 | |
| | Day 6 | | over control | | Lipid | |
| | Biomass | Lipid | % | Fold | production | |
| Treatments | (g L⁻¹) | (mg L⁻¹) | increase | increase | (mg L⁻¹) | % |
| Control | 0.268 | 13.4 | | | 13.4 | 5 |
| T1 | 0.268 | 13.4 | 75 | 1.75 | 23.5 | 9 |
| T2 | 0.268 | 13.4 | 115 | 2.15 | 28.8 | 11 |
| T3 | 0.268 | 13.4 | 336 | 4.36 | 58.4 | 22 |

This projection is based on harvesting cells on day 6 with biomass concentration of 0.268 g L⁻¹ with a lipid productivity of 13.4 mg L⁻¹ and treating the cells with various concentrations of malathion viz. 70 mg L⁻¹ (T1), 140 mg L⁻¹ (T2) and 280 mg L^{‘}(T3). As per the results the cells treated with malathion for 6 days showed 1.75, 2.15 and 4.36 times increase in lipid production over control for the treatments T1, T2 and T3, respectively. This projection indicates that the lipid content of the alga can be improved from 5% to 22% and lipid productivity from 13.4 mg L⁻¹ to 58.4 mg L⁻¹ (336% increase) through the treatment with

This projection was made considering the initial density of the cells as constant in control and other treatments. This projection is based on harvesting cells on day 6 with a biomass concentration of 0.268 g L⁻¹ with a lipid productivity of 13.4 mg L⁻¹ and treating the cells with various concentrations of malathion viz. 70 mg L⁻¹ (T1), 140 mg L⁻¹ (T2) and 280 mg L⁻¹ (T3). As per the results observed from the study the cells treated with malathion for 6 days showed 1.75, 2.15 and 4.36 times increase in lipid production over control for the treatments T1, T2 and T3, respectively. This projection indicates that the lipid content of the alga can be improved from 5% to 22% and lipid productivity from 13.4 mg L⁻¹ to 58.4 mg L⁻¹ (336% increase) through the treatment with malathion (280 mg L⁻¹) as stress stimulant after harvesting cells grown without stress (Table 3).

We claim:

1. A method of enhancing the biofuel potential of an algal culture, comprising:
providing a culture of at least one algal species;
contacting the algal culture with a composition selected to enhance the biofuel potential of an algal culture; and
allowing the algal culture to incubate for a time period, whereby the potential of the algal culture to provide a biofuel product or be processed to a biofuel product is enhanced compared to when the algal culture is not in contact with the composition.

2. The method according to claim 1, wherein the alga species is a species of a genus selected from the group consisting of: Gloeocystis, Limnothrix, Scenedesmus, Chlorococcum, Chlorella, Anabaena, Chlamydomonas, Botryococcus, Cricosphaera, Spirulina, Nannochloris, Dunaliella, Phaeodactylum, Pleurochrysis, Tetraselmis, and any combination thereof.

3. The method according to claim 1, wherein the alga species is Chlorella sorokiniana.

4. The method according to claim 1, wherein the composition selected to enhance the biofuel potential of an algal culture is selected from the group consisting of: an auxin, a phytohormone, a cytokinin, a cytokinin like compound, a growth promoter, a micronutrient, and any combination thereof.

5. The method according to claim 1, wherein the composition selected to enhance the biofuel potential of an algal culture is selected from the group consisting of: phenyl acetic acid indole-butryic acid, naphthalene acetic acid, gibberellic acid, zeatin, thidiazuron, humic acid, kelp extract, methanol, iron chloride, and any combination thereof.

6. The method according to claim 1, wherein the enhanced biofuel potential is selected from the group consisting of: the biomass of the algal culture and the lipid content of the algal culture.

7. The method according to claim 1, wherein the composition selected to enhance the biofuel potential of an algal culture is contacted with the algal culture at a time point within the incubation time period whereby enhancement of the biofuel potential of an algal culture is greater than when the composition is contacted with the algal culture at a different time point within the incubation time period.

8. The method according to claim 1, wherein the composition selected to enhance the biofuel potential of an algal culture is a pesticide.

9. The method according to claim 7, wherein the pesticide is malathion (2-(dimethoxyphosphinothioylthio)butanedioic acid diethyl ester).
